(19) United States
(12) Patent Application Publication
(10) Pub. No.: US 2020/0360711 A1
(43) Pub. Date: Nov. 19, 2020

(54) DELIVERY OF MAGNETIC PARTICLES IN CONJUNCTION WITH THERAPEUTIC AND/OR DIAGNOSTIC AGENTS

(71) Applicant: PULSE THERAPEUTICS, INC., St. Louis, MO (US)

(72) Inventors: Brian L. Kidd, St. Louis, MO (US); Francis M. Creighton, St. Louis, MO (US); Michael E. Sabo, St. Louis, MO (US); Sean C. Morris, St. Louis, MO (US)

(21) Appl. No.: 16/766,906

(22) PCT Filed: Nov. 27, 2018
(86) PCT No.: PCT/US2018/062610

§ 371 Date: May 26, 2020

Related U.S. Application Data
(60) Provisional application No. 62/591,989, filed on Nov. 29, 2017.

(51) Int. Cl. A61N 2/12; A61B 17/3207; A61B 34/00; A61N 2/00

(52) U.S. Cl. CPC A61N 2/12; A61B 17/320758; A61B 2017/00345; A61N 2/002; A61B 34/73

(57) ABSTRACT

Controlled delivery of magnetic particles (e.g., nanoparticles) in combination with therapeutic, diagnostic and/or theranostic agents is described. The magnetic particles may be delivered through a catheter to a location adjacent a therapeutic and/or diagnostic target or region.

### CROSS REFERENCE TO RELATED APPLICATIONS

This application claims priority to U.S. Provisional Application No. 62/591,989 filed Nov. 29, 2017, the entire content of which is hereby incorporated by reference herein. This application also generally relates to PCT Publication No. WO 2013/173235 published on Nov. 21, 2013, the entire content of which is hereby incorporated by reference herein.

### FIELD

This disclosure generally relates to systems and methods for facilitating introduction and external manipulation of magnetic particles (e.g., nanoparticles) within a body of a subject (e.g., within vasculature or other body lumens) for the treatment of various conditions. This disclosure also relates to systems and methods for facilitating controlled delivery of contrast media and/or therapeutic agents within the body of the subject in conjunction with the magnetic particles for therapeutic, diagnostic or combined diagnostic and therapeutic purposes.

### BACKGROUND

The treatment of fluid obstructions in the circulatory system, including vascular occlusions in vessels of the brain and vessels of the extremities, has included the use of drugs that can dissolve the obstructions and the use of obstruction removal devices, such as thrombectomy devices.

### SUMMARY

In accordance with several embodiments, a method of facilitating treatment of a therapeutic target (e.g., a clot, occlusion, obstruction, plaque, tumor) within a body lumen, passage, volume or space (e.g., blood vessel, tube, bone cavity, cerebrospinal column, heart, pericardium, lymphatic system, circulatory system) of a subject includes advancing a distal end of a catheter (e.g., microcatheter) to a location of the therapeutic target. The method also includes delivering magnetic particles (e.g., nanoparticles) through the catheter (e.g., microcatheter) at the location of (e.g., proximal to, distal to, or within) the therapeutic target (e.g., clot, thrombus). The method further includes delivering a therapeutic agent (e.g., thrombolytic, plasminogen, microplasmin, plasmin, theranostic agent) through the catheter at the location of the therapeutic target (e.g., clot, thrombus) and deploying a retrieval device (e.g., thrombectomy device) from the distal end of the catheter so as to capture the therapeutic target (e.g., clot, thrombus) with the retrieval device. The method also includes applying a rotating magnetic field so as to cause the magnetic particles (e.g., nanoparticles) to agglomerate into stir bars and to travel in a rotating motion. The stir bars and the therapeutic agent (e.g., thrombolytic agent) are adapted and positioned to travel with, and facilitate lysis of, any fragments (e.g., clot fragments, emboli) that break off from the therapeutic target (e.g., clot) during the deploying and withdrawing of the retrieval device (e.g., thrombectomy device).

The method may also include removing the retrieval device from the subject (e.g., withdrawing the thrombectomy device from the blood vessel). A thrombectomy device may comprise a self-expanding stent-like member comprised of shape-memory material or an inflatable member or a mechanically-expandable member adapted to remove (e.g., capture) a clot (e.g., thrombus, occlusion). The retrieval device (e.g., thrombectomy device) may also comprise a suction element or one or more suction ports adapted to facilitate capture of the clot via activation of an aspiration or vacuum source. Delivering the therapeutic agent (e.g., thrombolytic agent, plasminogen, microplasmin, neuroprotectant, cardioprotectant) through the catheter at the location of the therapeutic target (e.g., clot) may include delivering the agent at locations proximal to, within, and/or distal of the therapeutic target (e.g., clot). The magnetic particles (e.g., nanoparticles) may be delivered proximal to, within, and/or distal of the therapeutic target (e.g., clot, obstruction). Delivering distal to the therapeutic target may benefit stir bar formation due to little or no flow in that region. If the thrombectomy device incorporates aspiration or suctioning, the magnetic particles (e.g., nanoparticles) and/or therapeutic agent could be delivered while negative vacuum pressure or suctioning is being applied.

The method may further include delivering a diagnostic agent (e.g., contrast agent or contrast media) through the catheter at the location of the therapeutic target (e.g., clot, obstruction). The diagnostic agent may be attached to (e.g., coated with, conjugated to, attached to a coating of) one or more of the magnetic particles (e.g., nanoparticles). The method may include imaging the magnetic particles (e.g., nanoparticles) utilizing an imaging modality (e.g., magnetic resonance imaging, ultrasound-based imaging, fluoroscopy, tomography-based imaging, etc.). In some implementations, advancing the distal end of the catheter to the location of the therapeutic target comprises positioning a distal terminus of the catheter at a location distal of the therapeutic target (e.g., clot). The catheter may be advanced over a guidewire and through a lumen of a guide catheter. The therapeutic target may be within a cerebral vessel or a peripheral vessel (e.g., vessel of an extremity (e.g., limb, such as an arm or leg) of the subject. The vessel may be an artery or a vein. Applying the rotating magnetic field may be performed by a permanent magnet or an electromagnet positioned external to the subject. In implementation involving a permanent magnet, a position and/or orientation of the permanent magnet may be adjusted so as to cause the stir bars to travel to a desired location.

In accordance with several embodiments, a method of facilitating treatment and/or imaging of a therapeutic and/or diagnostic target within a body of a subject includes delivering magnetic particles (e.g., nanoparticles) to a location near the target within the body of the subject, applying a rotating magnetic field so as to cause the magnetic particles (e.g., nanoparticles) to agglomerate into stir bars and to travel in a rotating motion toward the target, and delivering one or more theranostic agents to the location near the target, wherein the travel of the stir bars in a rotating motion causes the one or more theranostic agents to reach the target (e.g., despite the presence of little or no fluid flow) and to remain at the target (e.g., despite the presence of fluid flow).

In some implementations, the one or more theranostic agents are attached (e.g., reversibly or irreversibly, directly or indirectly) to one or more of the magnetic particles (e.g., nanoparticles). Delivering the magnetic particles (e.g., nanoparticles) and delivering the one or more theranostic agents may comprise systemic intravenous delivery or may comprise local delivery through a catheter or other introducer to the location near the therapeutic target. The magnetic particles (e.g., nanoparticles) and the one or more theranostic agents may be mixed and/or co-administered together at the same time or delivered separately.

In accordance with several embodiments, a method of facilitating treatment and/or diagnosis of a therapeutic and/or diagnostic target within a body of a subject includes advancing a distal end of a catheter (e.g., microcatheter) to a location of the target within the body of the subject, introducing a plurality of magnetic particles (e.g., nanoparticles) within a lumen of the catheter, applying a rotating magnetic field so as to cause the magnetic nanoparticles to agglomerate into stir bars within the lumen of the catheter, delivering the stir bars and one or more therapeutic, diagnostic, and/or theranostic agents out of the catheter toward the location of the target, and continuously applying the rotating magnetic field for a duration of time such that the stir bars convey the one or more therapeutic, diagnostic, and/or theranostic agents (e.g., thrombolytics and/or plasminogen, microplasmin or plasmin, neuroprotectants, cardioprotectants, contrast agents) to the therapeutic target. The method may include delivering one or more diagnostic agents through the catheter separately or concurrently with one or more therapeutic agents. The one or more therapeutic agents may include one or more theranostic agents. The diagnostic, therapeutic and/or theranostic agents may be mixed with the magnetic particles (e.g., nanoparticles) prior to introduction or may be introduced separately or may be coated, doped with, or otherwise attached, conjugated or adsorbed to the magnetic particles.

In some implementations, applying the rotating magnetic field includes rotating a permanent magnet positioned external to the subject. For example, the permanent magnet may be positioned within a housing coupled to a portable base. The housing may be coupled to a suspension arm coupled to the portable base. The suspension arm may facilitate manipulation of the position of the housing so as to be placed adjacent a target region of a patient's body. The magnet may be configured to rotate about two or more axes of rotation. The method may further include adjusting a position and/or orientation of the permanent magnet so as to cause the stir bars to travel to a desired location. Rotating the permanent magnet may include rotating the permanent magnet at a frequency of between 0.1 and 100 Hz (e.g., between 1 Hz and 50 Hz, between 1 Hz and 10 Hz, between 5 Hz and 20 Hz, between 10 Hz and 30 Hz, between 15 Hz and 35 Hz, between 20 Hz and 40 Hz, between 25 Hz and 50 Hz, between 3 Hz and 6 Hz, overlapping ranges thereof, or any value within the recited ranges). The permanent magnet may have a magnitude between 0.01 and 1 Tesla (e.g., between 0.01 and 0.1 Tesla, between 0.01 and 0.05 Tesla, between 0.1 and 0.5 Tesla, between 0.5 Tesla and 1 Tesla, overlapping ranges thereof, and any value within the recited ranges). In some implementations, the rotating magnetic field is applied by rotating multiple permanent magnets positioned external to the body of the subject. In other implementations, the rotating magnetic field is generated by controlling currents through one or more electromagnets positioned external to the body of the subject.

The therapeutic and/or diagnostic target may be a clot or other fluid obstruction within a blood vessel such as a cerebral artery or pulmonary artery or a vessel (e.g., artery or vein) in a leg or other limb (e.g., arm) of an extremity of the subject. The therapeutic target may be a tumor or an obstruction within a Fallopian tube or a target (e.g., tumor) at an intraosseous location. The magnetic particles (e.g., nanoparticles) may be iron oxide (e.g., magnetite) nanoparticles. The magnetic particles may comprise superparamagnetic particles or ferromagnetic particles. The therapeutic agents may include an anti-cancer drug adapted to treat a tumor. In some implementations, the therapeutic target is an obstruction within an in-dwelling catheter. The therapeutic agents may include one or more thrombolytic drugs, plasminogen, microplasmin, plasmin, neuroprotectants, and/or cardioprotectants.

In accordance with several embodiments, a method of facilitating treatment of a therapeutic target within a body of a subject includes advancing a distal end of a catheter to a location of the therapeutic target within the body of the subject, introducing a plurality of magnetic nanoparticles within a lumen of the catheter, applying a rotating magnetic field using a permanent magnet positioned external to the subject so as to cause the magnetic nanoparticles to agglomerate into stir bars within the lumen of the catheter, imaging the stir bars within the lumen of the catheter, determining a desired direction of travel of the stir bars based on the imaging, adjusting at least one of a position and an orientation of the permanent magnet so as to cause the stir bars to travel in the desired direction of travel, delivering the stir bars and one or more therapeutic agents out of the catheter toward the location of the therapeutic target and continuously applying the rotating magnetic field for a duration of time such that the stir bars convey the one or more therapeutic agents to the therapeutic target, thereby facilitating treatment of the therapeutic target. Imaging the stir bars may include use of at least one of fluoroscopy, ultrasound-based imaging, magnetic resonance imaging, tomography-based imaging, or other imaging modalities or equipment. In some embodiments, multiple permanent magnets may be positioned at different external locations. The therapeutic target may be within a cerebral vessel or a peripheral vessel (e.g., vessel of an extremity (e.g., limb, such as an arm or leg) of the subject. The vessel may be an artery or a vein.

In accordance with several embodiments, a method of facilitating clearance of an obstruction within an in-dwelling catheter without removing the catheter from a subject includes introducing a plurality of magnetic nanoparticles within a lumen of the catheter, applying a rotating magnetic field using a permanent magnet positioned external to the subject so as to cause the magnetic nanoparticles to agglomerate into stir bars and to travel in a rotating end-over-end motion toward the obstruction, and delivering one or more therapeutic agents adapted to clear the obstruction into the lumen of the catheter, wherein the rotating end-over-end motion of the stir bars causes the one or more therapeutic agents to be conveyed to the obstruction such that the one or more the therapeutic agents can effectively clear the obstruction. The method may also include imaging the stir bars or the motion of the stir bars so as to identify a location of the obstruction. In some embodiments, multiple permanent magnets may be positioned at different external locations.

In accordance with several embodiments, a method of facilitating clearance of an obstruction within an in-dwelling catheter without removing the catheter from a subject includes introducing a plurality of magnetic nanoparticles within a lumen of the catheter, applying a rotating magnetic field using a permanent magnet positioned external to the subject so as to cause the magnetic nanoparticles to agglomerate into stir bars and to travel in a rotating end-over-end motion toward the obstruction, and imaging the stir bars or the motion of the stir bars so as to identify a location of the obstruction. The method may also include clearing the obstruction based on identified location (e.g., using one or more cleaning devices or by delivering one or more therapeutic agents adapted to clear the obstruction).

In accordance with several embodiments a system adapted to facilitate thrombectomy procedures with enhanced efficacy and safety includes a multi-lumen microcatheter having a primary lumen and a secondary lumen and a thrombectomy device adapted to be delivered through the primary lumen of the multi-lumen microcatheter. The thrombectomy device includes an expandable member adapted to capture and/or otherwise retrieve (e.g., remove) a clot (e.g., thrombus, obstruction, occlusion). The outer diameter of the microcatheter may be sized to fit within a cerebral blood vessel or other blood vessel of a subject. The system also includes a permanent magnet adapted to be positioned external to the subject and a magnetic controller adapted to rotate the permanent magnet so as to cause magnetic particles (e.g., nanoparticles) introduced within the secondary lumen of the microcatheter to agglomerate into stir bars and to travel in a rotating end-over-end motion, wherein the rotating end-over-end motion of the stir bars causes one or more therapeutic agents delivered through the catheter to remain at the location of the clot. In some embodiments, multiple permanent magnets may be positioned at different external locations to effect movement of the magnetic particles.

In accordance with several embodiments, the microcatheter facilitates delivery of magnetic particles (e.g., nanoparticles), therapeutic agents, diagnostic agents, and/or theranostic agents proximal to, within, or distal to the location of the clot through one or more separate lumens from the lumen used to introduce the thrombectomy device. In some embodiments, the microcatheter facilitates delivery of magnetic particles (e.g., nanoparticles) and therapeutic agents into the clot (e.g., thrombus, occlusion, obstruction) while an expandable retrieval member of the thrombectomy device is engaged with and is trapping the clot (e.g., through radial or side openings or apertures positioned along the microcatheter), thereby facilitating lysis of the clot and any distal emboli that break off in addition to just simply capturing and removing the clot. The microcatheter may include one or more structures or mechanisms (e.g., an occlusive member (e.g., plug or balloon) on an end of an elongate shaft or wire) configured to temporarily close a normally-open distal end of the microcatheter during the thrombectomy procedure to facilitate formation of agglomerated nanoparticle stir bars within the catheter prior to release from the microcatheter. At least a portion of the microcatheter may be transparent to one or more imaging modalities so as to facilitate imaging of the stir bars (which may be made opaque to a particular imaging modality) within the microcatheter.

In accordance with several embodiments, the magnetic particles (e.g., nanoparticles) may be used to enhance the flow, efficacy and ability to move contrast media (e.g., contrast agents, dyes) through tortuous vessels (either through systemic intravenous introduction without use of a catheter or through local introduction (e.g., intra-arterial introduction) using a catheter). The use of the magnetic nanoparticles (e.g., and formation of stir bars and causing rotating end-over-end motion of the stir bars) by applying a rotating magnetic field and directed gradient can advantageously result in less (e.g., 10-50% less) of the contrast media that is required to be used than without use of the magnetic nanoparticles. The use of less contrast media can advantageously reduce the likelihood of adverse consequences and undesired side effects resulting from delivery of excess contrast media, such as kidney failure, toxicity, deposits in the brain, bone, and other organs that do not tolerate significant amounts of contrast media. In addition, the use of the magnetic nanoparticles in the manner described herein may advantageously extend the imaging time at the target region (e.g., low/no flow area) by keeping the contrast media captured and concentrated in the target region for an extended period of time.

The methods summarized above and set forth in further detail below may describe certain actions taken by a practitioner; however, it should be understood that they can also include the instruction of those actions by another party. For example, actions such as “advancing a microcatheter” include “instructing the advancement of a microcatheter.” Further aspects of embodiments of the inventions will be discussed in the following portions of the specification. With respect to the drawings, elements from one figure may be combined with elements from the other figures.

### BRIEF DESCRIPTION OF THE DRAWINGS

The drawings, which are briefly described below, are for illustrative purposes only. The drawings are not intended to limit the scope of the disclosure in any way and may not be to scale.

FIG. 1 schematically illustrates a sequence showing agglomeration of magnetic particles into an agglomerated structure under the influence of an applied magnetic field.

FIGS. 2A and 2B schematically illustrate an agglomerated structure rotating and translating as a result of a time-varying magnetic field and a gradient.

FIG. 2C schematically illustrates movement of multiple agglomerated structures toward a clot within a blood vessel.

FIGS. 3A and 3B illustrate introduction of magnetic particles into a region of low flow through an endovascular catheter and application of a time-varying magnetic field to the magnetic particles.

FIGS. 4A and 4B illustrate use of a balloon to temporarily occlude blood flow within a blood vessel and then introducing magnetic particles into a region of low flow through an endovascular catheter and applying a time-varying magnetic field to the magnetic particles.

FIGS. 5A-5D illustrate introduction of magnetic particles through a dual-lumen catheter in conjunction with a thrombectomy procedure.

FIGS. 6A-6E illustrate the introduction of magnetic particles through a dual-lumen catheter in conjunction with a thrombectomy procedure, as well as use of a magnet to cause the magnetic particles to assemble into agglomerates inside the catheter before being released from the catheter into the blood vessel

FIG. 7 illustrates a distal end portion of an embodiment of a microcatheter and schematically illustrates introduction of magnetic particles and thrombolytic through the catheter to locations within, proximal and/or distal of a clot or other obstruction.

FIGS. 8A-8D illustrate various cross-section views along various embodiments of catheters adapted to facilitate introduction of magnetic particles and/or thrombolytic to locations within, proximal and/or distal of a clot or other obstruction.

FIGS. 9A-9C and 10A-10C schematically illustrate methods of determining and adjusting alignment and position of a magnet positioned external to a body of a subject.

FIG. 11 schematically illustrates how direction of travel of the magnetic particles can be altered to more efficiently move the magnetic particles along various portions or lengths of vasculature.

FIG. 12 schematically illustrates that various regions of vasculature can be identified and associated with different magnet positions and orientations to make travel of the magnetic particles more efficient to access vasculature within the various regions.

FIGS. 13A-13G illustrate an example user interface for use in treating a patient with magnetic nanoparticles and a magnetic control system.

FIGS. 14A and 14B illustrate an embodiment of a magnetic control system and the positioning of a magnet pod of the magnetic control system with reference to a patient being treated for potential obstruction or blockage in a brain vessel.

### DETAILED DESCRIPTION

### Abbreviations and Definitions

The scientific and technical terms used in connection with the disclosure shall have their ordinary meanings (e.g., as commonly understood by those of ordinary skill in the art) in addition to any definitions included herein. Further, unless otherwise required by context, singular terms shall include pluralities and plural terms shall include the singular.

“Patient” or “subject” shall be given its ordinary meaning and shall include, without limitation, human and veterinary subjects.

“Thrombolytic” shall be given its ordinary meaning and shall include, without limitation, drugs or compositions capable of degrading a blood clot or atherosclerotic plaque. For example, a thrombolytic drug can include tissue plasminogen activator (tPA), plasminogen, streptokinase, urokinase, recombinant tissue plasminogen activators (rtPA), alteplase, reteplase, tenecteplase, and other drugs, and can include these drugs administered alone or co-administered with warfarin and/or heparin. Different thrombolytic drugs can be used in the thrombolytic process. For example, streptokinase can be used in some cases of myocardial infarction and pulmonary embolism. Urokinase can be used in treating severe or massive deep venous thrombosis, pulmonary embolism, myocardial infarction and occluded intravenous or dialysis cannulas. Tissue Plasminogen Activator (“tPA” or “PLAT”) can be used clinically to treat stroke. Reteplase can be used to treat heart attacks by breaking up the occlusions that cause them.

In the case of stroke (e.g., cardioembolic stroke or acute ischemic stroke), tPA is used successfully in many cases, but in many cases the effect of the drug is to leave downstream residue in clumps large enough to cause further blockage and sometimes death. In addition, the normal thrombolytic dosage administered to patients is related to increased bleeding in the brain. In many cases, the effectiveness of chemical interaction of the thrombolytic agent with the blockage is slow and inefficient, leaving incomplete removal of the blockage. In blockages in the extremities, mechanical means of stirring and guiding the drug are limited, often difficult, and can be dangerous. In many cases, venous valves in the region of the procedure are damaged or not made blockage-free in procedures currently used. Some embodiments described herein advantageously provide new systems and methods for significant improvements in dealing with these major obstacles in treating occlusions of the blood flow.

“Magnetic particle” shall be given its ordinary meaning and shall include, without limitation, magnetic nanoparticles having a diameter greater than or equal to about 1 nm and/or less than or equal to about 1000 nm, greater than or equal to about 10 nm and/or less than or equal to about 200 nm, greater than or equal to about 15 nm and/or less than or equal to about 150 nm, greater than or equal to about 20 nm and/or less than or equal to about 60 nm, 80 nm, 100 nm, and all integer values between 1 nm and 1000 nm, e.g., 1, 2, 3, 4, 5, . . . 997, 998, 999, and 1000. The appropriate sizes of magnetic particles can depend on the therapeutic target of the system (e.g., very small vessels can accept smaller nanoparticles and larger parts of a circulatory system can accept larger nanoparticles). Examples of such magnetic particles include ferrimagnetic iron oxide nanoparticles or super-paramagnetic nanoparticles. The particles may be made of magnetite or other ferromagnetic mineral or iron oxide and, in some embodiments, can be co-administered, coated or conjugated with any one or a combination of the following materials: (1) coatings which enhance the behavior of the nanoparticles in blood by making them either hydrophilic or hydrophobic; (2) coatings which buffer the nanoparticles and which optimize the magnetic interaction and behavior of the magnetic nanoparticles; (3) contrast agent or agents which allow visualization with magnetic resonance imaging, X-ray, Positron Emission Tomography (PET), ultrasound, or other imaging technologies; (4) therapeutic agents which accelerate destruction of a circulatory system blockage; (5) stem cells; (6) chemotherapeutic drugs; and (7) thrombolytic drugs. The term “magnetic nanoparticle” when used herein can be substituted with “magnetic particle” so as to include microparticles or other larger particles.

“Fluid obstruction” shall be given its ordinary meaning and shall include, without limitation, a blockage, either partial or complete, that impedes the normal flow of fluid through a circulatory system, including the venous system, arterial system, central nervous system, and lymphatic system. “Vascular occlusions” are fluid obstructions that include, but are not limited to, atherosclerotic plaques, fatty buildup, arterial stenosis, restenosis, vein thrombi, cerebral thrombi, embolisms (e.g., pulmonary embolisms), hemorrhages, other blood clots, and very small vessels. Sometimes, fluid obstructions are generally referred to herein as “clots.”

“Embolus/Emboli” shall be given its ordinary meaning and shall include, without limitation, fragments or portions of a clot or thrombus that break off from a main body of the clot or thrombus.

“Contrast Agent” shall be given its ordinary meaning and shall include, without limitation, any material (solid or liquid) that facilitates visualization or imaging utilizing any imaging modality.

“Increased fluid flow” shall be given its ordinary meaning and shall include, without limitation, increasing the throughput of a blocked or occluded body lumen from zero to something greater than zero. For example, in flowing circulatory systems, the term increased fluid flow can mean increasing the throughput from a level prior to administration of one or more magnetic particles in a patient to a level greater than the original fluid flow level.

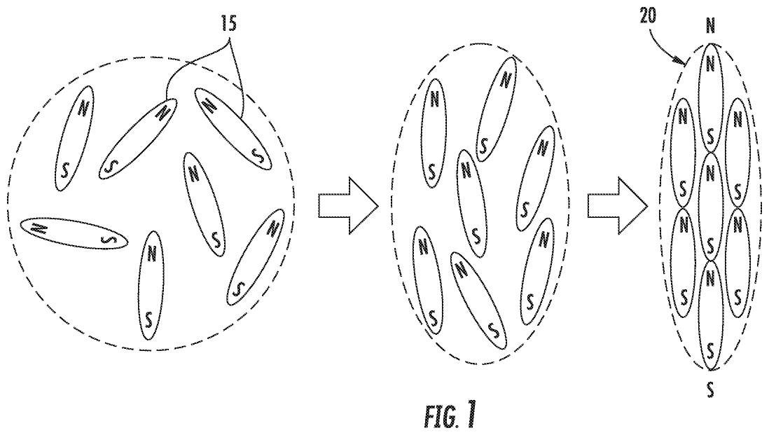

“Agglomerate” shall be given its ordinary meaning and shall include, without limitation, rotational clustering and chaining of a group of individual magnetic particles (e.g., nanoparticles) in a manner to form “stir bars” or “stir rods” from the magnetic particles (for example, as described herein with respect to FIG. 1), as well as the combined structures themselves when used as a noun.

“Treatment” shall be given its ordinary meaning and shall include, without limitation, an approach for obtaining beneficial or desired clinical results. For purposes of this disclosure, beneficial or desired clinical results include, but are not limited to, one or more of the following: improvement or alleviation of any aspect of fluid obstruction within a body of a subject or within a device including, but not limited to, fluid obstructions (e.g., stroke, deep vein thrombosis, myocardial infarction), coronary artery disease, peripheral artery disease, ischemic heart disease, limb ischemia (e.g., atherosclerosis), internal bleeding, and high blood pressure; cancer treatment; or movement along any body lumen, space or cavity to access a desired treatment target.

Several embodiments of the inventions are particularly advantageous because they include one, several or all of the following benefits: (i) delivery of diagnostic or therapeutic and/or theranostic agents to target locations even with little or no flow; (ii) keeping diagnostic or therapeutic and/or theranostic agents at a target location longer than conventional approaches even when there is flow that would normally cause the agents to flow away; (iii) use of less contrast media than conventional techniques require, thereby reducing adverse effects due to exposure to large amounts of contrast media, such as kidney failure; (iv) more efficient travel of therapeutic or diagnostic and/or theranostic agents to target locations; (v) prioritization of target treatment locations; (vi) identification of locations and/or treatment of distal emboli created during thrombectomy procedures; (vii) identification of and/or clearance of obstructions within in-dwelling catheters; (viii) delivery of magnetic particles without having them pass through the lungs; and/or (ix) reduced likelihood of adverse hemodynamic effects.

### Overview of Mechanism of Action

Systems and methods for the physical manipulation of magnetic particles (e.g., nanoparticles) within body lumens (e.g., vasculature) of a subject to facilitate clearance of fluid obstructions are described and illustrated in WIPO Publication No. 2011/053984 and in WIPO Publication No. 2013/173235, the entire contents of each of which are hereby incorporated by reference herein. The embodiments disclosed herein may be combined with and incorporated in conjunction with any of the embodiments or features of the magnetic control systems, therapeutic targets, or imaging or diagnostic methods disclosed in WIPO Publication No. 2013/173235, the entire content of which is hereby incorporated by reference herein.

Some embodiments of the invention relate to the control of magnetic particles (e.g., nanoparticles) to increase contact of a therapeutic target (e.g., clot, thrombus, occlusion, obstruction) in a portion of a circulatory system (e.g., artery, vein) with a therapeutic agent (e.g., a pharmaceutical compound, a thrombolytic drug, microplasmin, plasmin or naturally-occurring thrombolytic within the body such as plasminogen, neuroprotectant, cardioprotectant), which can result in increased fluid flow and the substantial clearance of fluid blockages, or obstructions, of body lumens (e.g., vasculature, blood vessels, organs, tubes, canals). In various aspects, the systems and methods described herein advantageously enhance diffusion of one or more therapeutic, diagnostic or combined therapeutic and diagnostic (theranostic) agents or delivery of the therapeutic, diagnostic or theranostic agents to a region of low or no flow. Magnetic fields and gradients can be used to act on magnetic nanoparticle agglomerates (e.g., stir bars or stir rods) to travel to desired treatment or diagnostic locations and/or to reduce obstructions or blockages, including vascular occlusions, in a patient. In various aspects, the system and methods described herein can be used to treat fluid blockages of the circulatory system in the head (in particular, the brain) and vessels within and surrounding the heart and in the extremities of the body, such as the vasculature of limbs (e.g., arms and legs). In various aspects, the system and methods described herein can be used simply to transport therapeutic agents through or along body passages that are difficult to access or traverse in an invasive or minimally invasive approach or to keep the therapeutic, diagnostic or theranostic agents in place for an extended period of time before they are washed downstream due to ordinary fluid flow. In some implementations, tissue plasminogen activator and one or more of plasminogen, microplasmin and plasmin are delivered to a clot to facilitate enhanced lysis efficacy. In other implementations, pro-coagulant materials (e.g., thrombin) and/or fibrinogen may be delivered using the magnetic particles so as to create clots at targeted, isolated locations so as to prevent internal bleeding at the locations. The magnetic particles may be coated or packaged together with the pro-coagulant materials. The pro-coagulant materials may act on the fibrinogen (e.g., delivered separately after localization of the pro-coagulant materials by control of the rotating magnetic field) to create the clots.

In some embodiments, a rotating magnetic field is generated by mechanically rotating a strong permanent magnet having an orientation that rotates the field at a target site, and at the same time presents a steady magnetic gradient in a desired direction. Rotational frequencies (e.g., greater than or equal to 0.1 Hz and/or less than or equal to 100 Hz, including but not limited to from about 1 Hz to about 30 Hz, from about 3 Hz to about 10 Hz, from about 0.5 Hz to about 50 Hz, from about 1 Hz to about 6 Hz, from about 0.1 Hz to about 10 Hz, from about 5 Hz to about 20 Hz, from about 10 Hz to about 30 Hz, from about 20 Hz to about 50 Hz, from about 40 Hz to about 70 Hz, from about 50 Hz to about 100 Hz, overlapping ranges thereof, less than 5 Hz, less than 10 Hz, less than 20 Hz, less than 30 Hz, less than 40 Hz, less than 50 Hz) can be effective with a range of magnetic field magnitudes that can be generated by magnets (e.g., greater than or equal to 0.01 Tesla and/or less than 1 Tesla, including but not limited to from about 0.01 Tesla to about 0.1 Tesla, from about 0.05 Tesla to about 0.5 Tesla, from about 0.1 Tesla to about 0.6 Tesla, from about 0.3 Tesla to about 0.9 Tesla, from about 0.5 Tesla to about 1 Tesla, overlapping ranges thereof, less than 1 Tesla, less than 0.5 Tesla, less than 0.25 Tesla, less than 0.1 Tesla). Gradient strength can be greater than or equal to 0.01 Tesla/m and/or less than or equal to 10 Tesla/m, including but not limited to from about 0.01 Tesla/m to about 1 Tesla/m, from about 0.01 Tesla/m to about 3 Tesla/m, from about 0.05 Tesla/m to about 5 Tesla/m, from about 1 Tesla/m to about 4 Tesla/m, overlapping ranges thereof, less than 5 Tesla/m, less than 3 Tesla/m, less than 2 Tesla/m, less than 1 Tesla/m). The gradient direction generally centers on the center of mass for a permanent magnet. In some embodiments, multiple permanent magnets may be positioned at different external locations and used to convey or propagate stir bars or stir rods or other agglomerated structures along a path. In some embodiments, the magnetic field may be created by controlling currents within an electromagnet.

When a magnetic field is imposed on a collection of magnetic particles (e.g., nanoparticles), they can combine, or assemble, to form larger structures (e.g., agglomerates or agglomerated structures or ensembles or stir bars or stir rods). The size of these assembled structures can be related to an applied magnetic field strength, a size of the magnetic particles (e.g., nanoparticles), and/or a thickness of an optional coating on the magnetic particles (e.g., nanoparticles). FIG. 1 illustrates agglomeration of magnetic nanoparticles 15 into an assembled structure (e.g., a stir rod or stir bar or spheroid) 20 as a result of the applied magnetic field. The magnetic nanoparticles 15 can become magnetized and align due in part to the applied magnetic field. As the applied magnetic field increases in strength, the magnetic nanoparticles 15 can continue to become magnetized and align, assembling into a larger structure, such as the rod 20 depicted in FIG. 1. At a certain rotating magnetic field strength and field rotation frequency, depending on nanoparticle size and coating, the rods 20 will reach a saturation field and achieve a maximum length. In one embodiment, for uncoated magnetite nanoparticles, the particles are close to a saturation point when the applied magnetic field is approximately 0.2 T. In some embodiments, nanoparticle size can affect the strength and/or rigidity of the assembled structure. For example, when an assembled structure has an angular momentum, a likelihood that the assembled structure (e.g., rod) 20 will break apart is inversely related to the size of the magnetic nanoparticles 15 making up the assembled structure 20. Fully developed agglomerates 20 may contain a number of nanoparticles, as many as ten or many more, depending on their size, and the magnitude of the rotating magnetic field. The agglomerates 20 are not stiff, depending on the magnetic field and gradient, and on the amount of magnetite in each nanoparticle 15 as well as the nanoparticle size.

In one example, a field of about 0.02 Tesla at the target site, in combination with a gradient of about 0.4 Tesla/meter, can create an agglomeration of magnetic nanoparticles (e.g., separated nanoparticle “stir rods” or “stir bars”). In general, the agglomerated structures (e.g., stir rods or stir bars) 20 can have a length that is greater than or equal to about 0.05 mm and/or less than or equal to about 3 mm in length, including but not limited to from about 0.05 mm to about 2 mm, from about 0.1 mm to about 2 mm, from about 0.2 mm to about 1.5 mm, from about 0.2 mm to about 1 mm, from about 0.3 mm to about 0.9 mm, from about 0.4 mm to about 0.8 mm, overlapping ranges thereof, less than 3 mm, less than 2 mm, less than 1.5 mm, less than 1 mm.

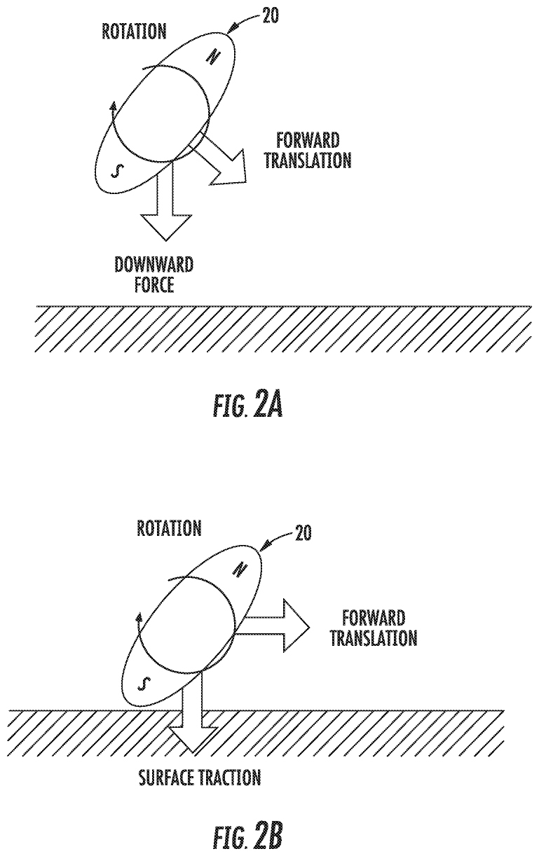

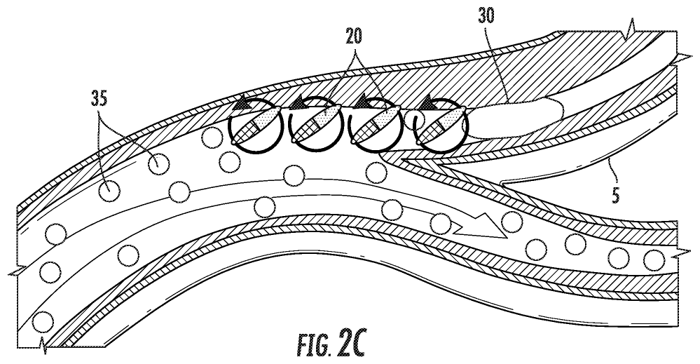

FIG. 2A illustrates an assembled structure 20, such as a stir rod or stir bar, rotating and translating as a result of a time-varying magnetic field. In some embodiments, the time-varying magnetic field can rotate and can have a magnetic field gradient. This combination can result in a torque and a net force on the agglomerated structure. Due in part to the torque, the stir rod or stir bar 20 can rotate. The rotation and the net force can result in a forward translation of the agglomerated structure 20 as illustrated.

FIG. 2B illustrates an agglomerated structure 20 rotating and translating across a surface as a result of a time-varying magnetic field. If the agglomerated structure 20 comes into contact with a surface, a combination of the torque, force from the magnetic gradient, and friction between the agglomerated structure 20 and the surface can result in a forward translation. The motion of the agglomerated structure 20 can be end-over-end, similar to an ellipse or spheroid rolling along a surface.

As described with respect to FIGS. 2A and 2B, the agglomerated structure 20 can rotate and translate as a result of a time-varying magnetic field having a gradient. The stir rod or stir bar 20 can rotate and translate in a forward direction when in contact with a surface, to the right in FIG. 2B. Due in part to the rotation and translation of the agglomerated structures 20, a flow can be generated in a surrounding fluid. As the agglomerated structure 20 moves (e.g., translates) forward it can experience a change in magnetic field. In some embodiments, the magnetic field can diminish with translation distance. As the gradient diminishes, the downward force on the agglomerated structure 20 can diminish. If the force diminishes past a threshold value, the agglomerated structure 20 can cease to be in contact with the surface, resulting in no friction force between the surface and the structure 20. The structure 20 can then experience a pressure arising from a flow of the fluid medium which surrounds the structure 20. This flow can result in a translation that is roughly backward, or left in FIG. 2B. As the structure 20 moves backward, the magnetic field gradient which the structure 20 experiences can increase and the structure 20 can be pulled back to the surface. Once back to the surface, the structure 20 can move forward in an end-over-end manner as explained above. The overall motion of the structure 20 can be generally circular or elliptical in nature. The end-over-end motion can facilitate travel of the structures 20 over complex terrains or surfaces within a patient's body.

With reference to FIG. 2C, in some embodiments, this flow pattern can increase mixing of a therapeutic and/or diagnostic agent (e.g., a thrombolytic, plasminogen, contrast agent, and/or theranostic agent or compound) or increase exposure of a therapeutic target (e.g., a clot, a tumor) to a therapeutic agent. In some aspects, the fluid can be a mixture of blood and a therapeutic agent (e.g., a thrombolytic drug), the blood and therapeutic agent being mixed by the generally circular motion of the agglomerated structures 20 to erode (e.g., lyse) and clear the therapeutic target. FIG. 2C illustrates how the movement of the agglomerated structures 20 can cause thrombolytic particles 35 to be “carried” or transported toward a fluid obstruction (e.g., clot) 30 even when there is little or no flow in a portion of a branch vessel 5 adjacent to the fluid obstruction 30.

By alternating a rotational direction of the magnetic stator system, the operator can direct the agglomerated structures (e.g., magnetic rotors) within a vessel. For example, within a vessel, a velocity of blood increases with distance from the vessel wall, where the velocity is approximately zero. A clotted vessel branch will obstruct fluid flow resulting in the velocity dropping to zero at the opening of the branch. Within such low velocity regions, magnetic nanoparticles generally assemble to be controlled by the magnetic stator system. When assembled, the magnetic stator system can agglomerate the magnetic nanoparticles into larger structures (e.g., magnetic rotors having an oblong shape). With a varying magnetic field, the magnetic rotors can rotate, resulting in an end-over-end motion that results in the magnetic rotors traveling into or next to the blocked branches. The resulting rotational motion of the magnetic rotors can create new currents or increase low-velocity currents. The resulting currents can concentrate a therapeutic agent in an otherwise inaccessible or difficult to access region. By changing the rotation of the magnetic stator system, additional branches can be infused. For example, different rotational directions can result in the magnetic rotors traveling to different branches. Rotational directions can be alternated to direct, or steer, magnetic rotors to multiple branches. In accordance with several embodiments, the magnetic rotors need not contact the therapeutic target to treat (e.g., reduce, erode, clear, or otherwise address) the target. For example, the magnetic rotors can facilitate treatment (e.g., removal or erosion) of a thrombus or clot without scraping or contacting the clot or occlusion. In some embodiments, the magnetic rotors infiltrate the target and deliver attached payload to the target.

In various embodiments, clots or thrombi of sizes larger than can be effectively treated by drug treatment (e.g., tPA) alone can be treated more efficiently (e.g., faster and/or with improved lysis) with the methods and systems described herein. For example, clots or thrombi having a cross-sectional dimension of 8 mm, 9 mm, 10 mm or greater than 10 mm (e.g., between 8 mm and 20 mm) can be effectively treated (e.g., lysed, dissolved, removed). In various embodiments, use of the methods and systems described herein can treat clots that have a near-zero or very little likelihood of being lysed (e.g., recanalizing occluded vessels) using tPA or other thrombolytic agent alone, such as clots or thrombi having lengths greater than 8 mm.

The treatments described herein can be effective even for patients deemed to have no likelihood of recanalization based on CMR or NIHSS scores if tPA or other thrombolytic agent alone were to be administered or patients suffering severe stroke as indicated by high NIHSS scores. In accordance with several embodiments, the magnetic nanoparticles do not aggravate tPA-induced hemorrhage. Introduction of Magnetic Particles Through a Catheter

In some embodiments, magnetic particles (e.g., nanoparticles) are administered to a location near (e.g., proximate, adjacent) a therapeutic target or fluid obstruction through a catheter (e.g., a microcatheter). For example, a catheter can be introduced intra-arterially and advanced to a location adjacent a clot within a cerebral artery or a peripheral artery. In accordance with several embodiments of the invention, introduction of the magnetic particles through the catheter advantageously reduces the amount of magnetic particles that need to be administered compared to a general, systemic intravenous administration. Introduction through a catheter may also avoid the magnetic particles having to travel through various organs (e.g., the lungs), where filtration may remove many of the magnetic particles, and may reduce the likelihood or opportunity for other biological reactions to take place before the magnetic particles reach a desired target location.

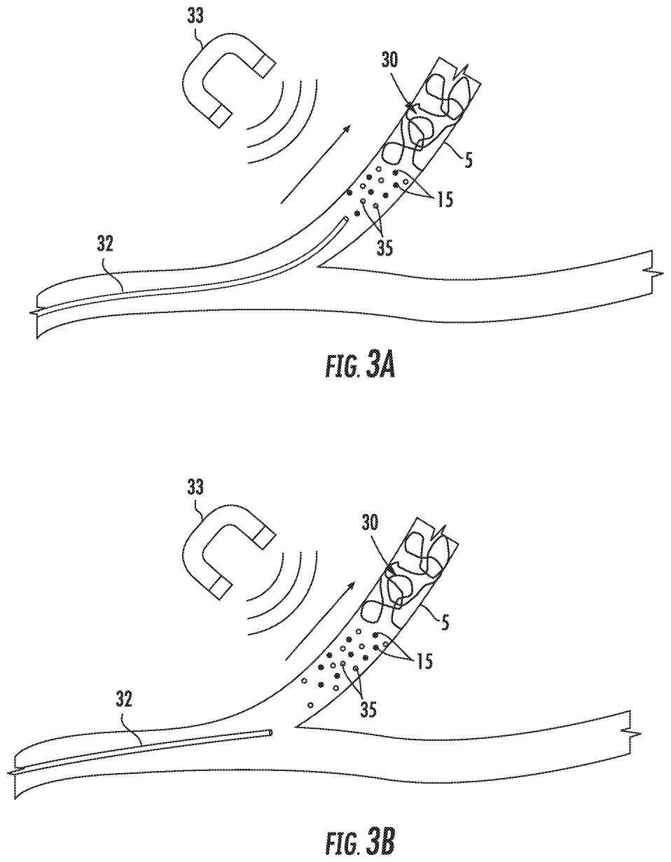

With reference to FIGS. 3A and 3B, in accordance with some embodiments, magnetic nanoparticles are introduced through a catheter (e.g., microcatheter) 32 directly to a region of low flow or no flow (e.g., a region adjacent a clot or thrombus 30 or build-up of plaque within a blood vessel). A magnetic field may then be applied using an external magnet 33 to cause the magnetic nanoparticles 15 to assemble into agglomerated structures 20 in the region of low flow or no flow instead of having a majority of the particles get washed down stream right away (which would normally occur under normal general intravenous injection). The external magnet 33 may be comprise one or more permanent magnets or an electromagnet system. The magnetic nanoparticles 15 could also be introduced into or near a vessel wall or any other location that allows the magnetic nanoparticles 15 to have time to assemble into agglomerates 20 (e.g., stir bars, stir rods) before being washed away by fluid flow. In some embodiments, the magnetic nanoparticles 15 are intentionally delivered through the catheter 32 and into or near the wall of a blood vessel, as there is less flow near the wall than at the center of the blood vessel, thereby facilitating more effective agglomeration (e.g., stir bar formation). Therapeutic and/or diagnostic fluid or materials 35 (e.g., thrombolytic agents, plasminogen, theranostic agents or compounds or contrast media) may also be introduced through the catheter 32, through a different introducer, or systemically via intravenous infusion, or the like.

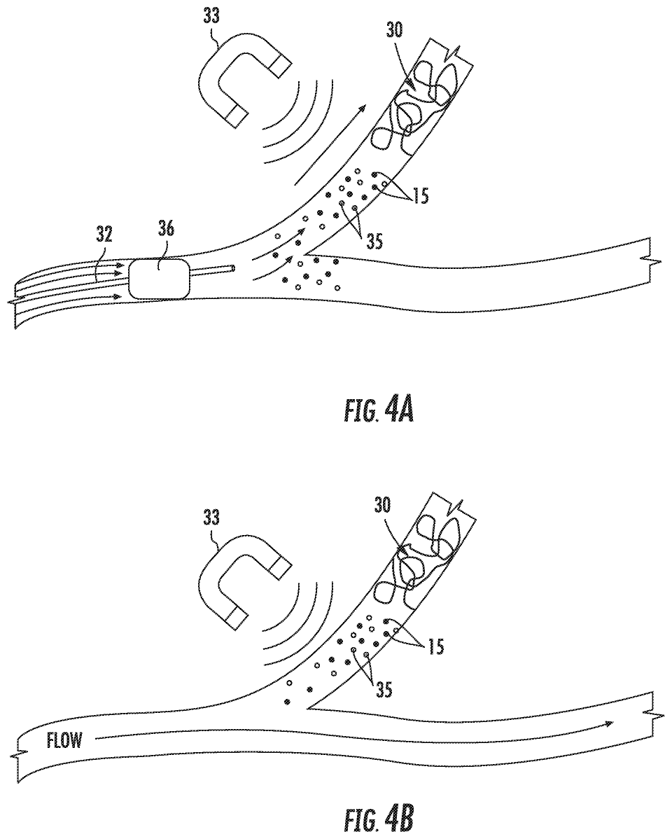

Turning to FIGS. 4A and 4B, the catheter 32 may include an occlusive member (e.g., balloon) 36 positioned along a distal end portion of the catheter 32. The occlusive member 36 may be deployed within a body lumen (e.g., blood vessel) to temporarily block flow while magnetic nanoparticles 15 and therapeutic and/or diagnostic fluid or materials 35 (e.g., thrombolytic agents, plasminogen, and/or contrast media) are delivered through the catheter into a target region of little or no flow caused by a fluid obstruction 40 (e.g., clot, thrombus, plaque, tumor). The deployment of the occlusive member 36 may advantageously allow the nanoparticles 15 an opportunity to assemble into agglomerates or stir bars at or adjacent the target region rather than immediately being washed downstream due to fluid flow. A rotating magnetic field and a magnetic gradient may be applied using an external magnet 33 to cause the magnetic nanoparticles 15 to assemble into agglomerates and then to travel (e.g., in an end-over-end manner or motion) toward the target region and the fluid obstruction 40. After a sufficient period of time for assembly of the magnetic nanoparticles 15 into agglomerates, the occlusive member 36 can be returned to an undeployed configuration and the catheter 32 can be removed so that flow can be restored. The magnet 33 may continue to be rotated for a desired treatment time. An electromagnet may alternatively be used to generate the magnetic field. Introduction of Magnetic Particles in Conjunction with Thrombectomy Procedures

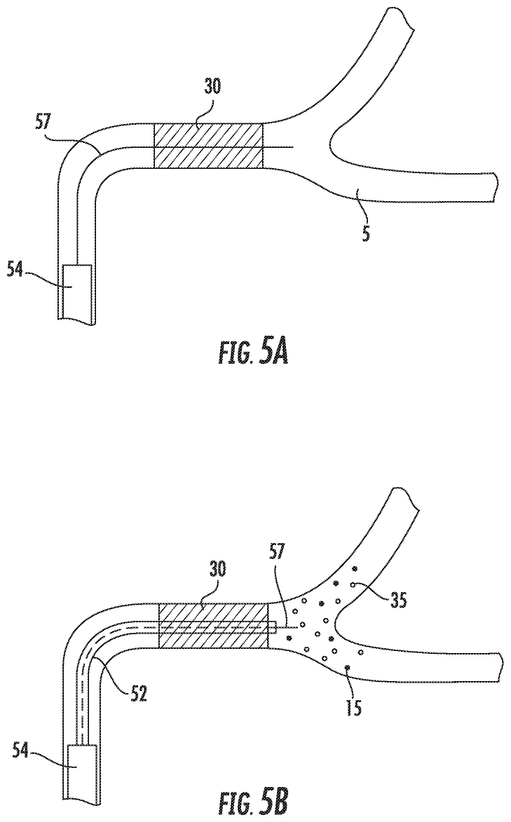

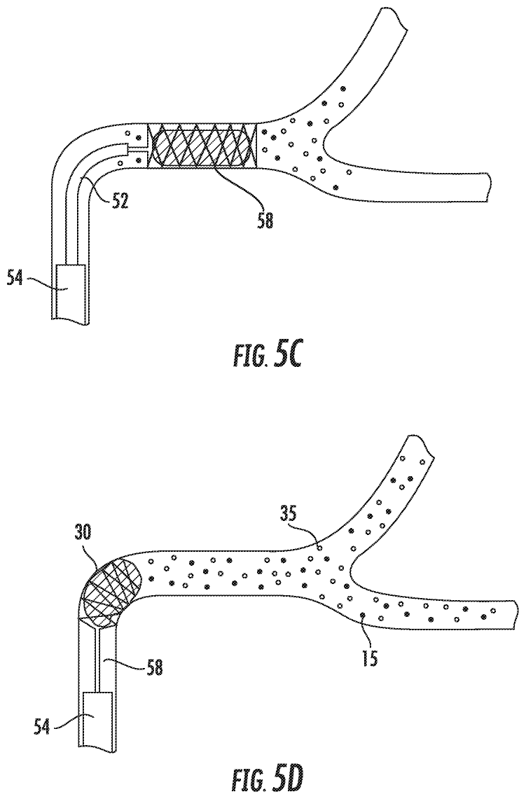

With reference to FIGS. 5A-5D, the magnetic particles (e.g., nanoparticles 15) may be introduced through a microcatheter 52 in conjunction with a thrombectomy procedure. FIGS. 5A-5D schematically illustrate various steps of an embodiment of the thrombectomy procedure. In FIG. 5A, a guide catheter 54 is advanced intravascularly to a location upstream of a thrombus, occlusion, or clot 30 that is occluding (at least partially) a blood vessel (e.g., a cerebral artery, peripheral artery). FIG. 5A also shows that a guidewire 57 has been delivered through the clot 30 such that a distal end of the guidewire 57 extends beyond (e.g., distal) of the clot 30. The guide catheter 54 may be advanced over the guidewire 57.

With reference to FIG. 5B, a microcatheter 52 is advanced out of a distal end of the guide catheter 54 and over the guidewire 57 until a distal end of the microcatheter 52 extends distal of the clot 30. In some embodiments, magnetic particles (e.g., nanoparticles 15) and/or thrombolytic materials 35 (e.g., tPA, streptokinase, urokinase, plasminogen, microplasmin, and/or plasmin) may be delivered through the microcatheter 52 distal to the clot 30.

With reference to FIG. 5C, a thrombectomy device (e.g., clot retriever) 58 is delivered out of the guide catheter 54 or the microcatheter 52 at the location of the clot 30. The guide catheter 54 or the microcatheter 52 may be withdrawn proximally to deploy the thrombectomy device 58 across the clot 30 (for example, if the thrombectomy device 58 comprises self-expanding or shape memory material). In some embodiments, magnetic particles (e.g., nanoparticles) and/or thrombolytic materials are delivered through the microcatheter 52 within the clot 30 and/or proximal to the clot 30 as the microcatheter 52 is withdrawn to further enhance lysis of the clot 30.

With reference to FIG. 5D, the thrombectomy device 58 (along with the captured clot 30) is withdrawn and removed from the blood vessel. Clot fragments (e.g., emboli) often break off from the clot during the thrombectomy procedure and during removal of the thrombectomy device 58 from the vasculature. The nanoparticles 15 and the thrombolytic materials 35 remain to address (e.g., lyse) any clot fragments (e.g., emboli) that have broken off or that break off during the thrombectomy procedure and during removal of the thrombectomy device 58 with the captured clot 30. In accordance with several embodiments, the nanoparticles 15 are opaque to an imaging modality (either due to the composition of the nanoparticles themselves or because contrast is mixed with, doped into, or conjugated or adsorbed to the nanoparticles) and can facilitate visualization of the boundaries of the clot 30. Imaging-opaque particles (e.g., nanoparticles) may also float downstream with the distal emboli or clot fragments and can thus be used to facilitate determination of the location of the distal emboli and subsequent treatment may advantageously be directed to the determined locations of the distal emboli (e.g., based on imaging of the particles).

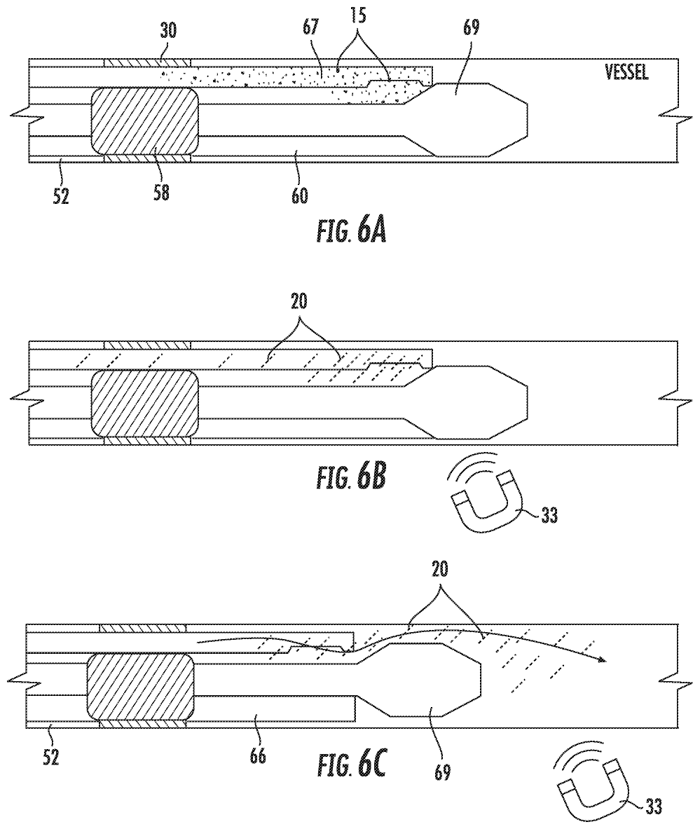

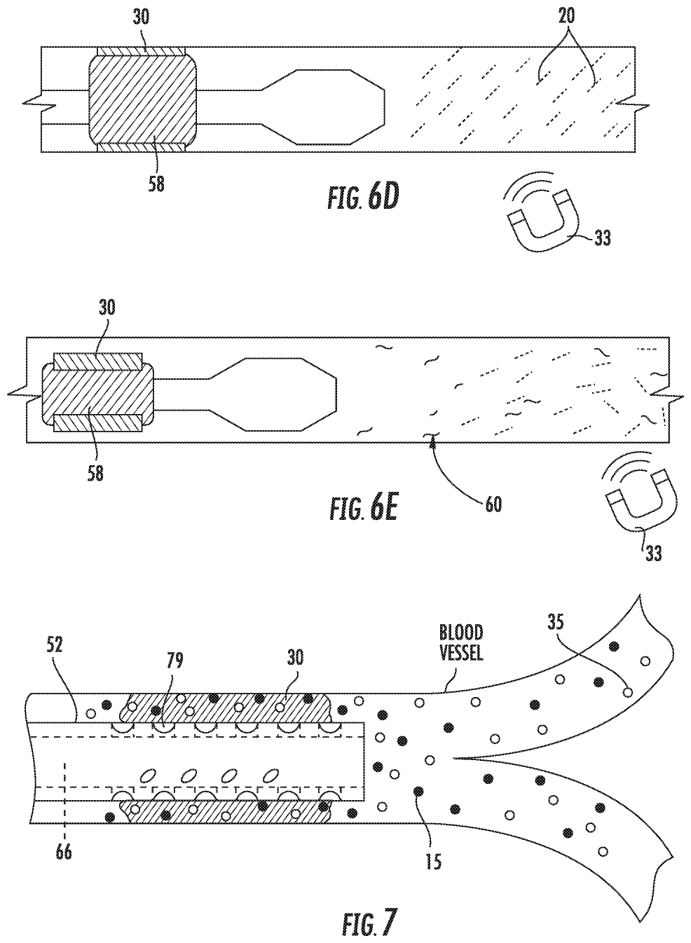

Turning to FIGS. 6A-6E, it may be advantageous in accordance with several embodiments for the magnetic particles (e.g., nanoparticles) 15 to be formed into agglomerates or stir bars while still within the catheter 52 or other introducer and before being delivered out of the catheter 52 so that the flow patterns created by the movement of the rotating agglomerates go into effect as soon as the magnetic nanoparticles 15 leave, or exit, the catheter 52. In some embodiments, this “pre-agglomeration” or “priming” allows more of the therapeutic, diagnostic or theranostic materials to remain at or close to a therapeutic, diagnostic or theranostic target.

FIG. 6A schematically illustrates an embodiment of the catheter 52 positioned within a blood vessel. In accordance with several embodiments, the catheter 52 is a microcatheter having multiple lumens (e.g., two lumens, three lumens, four lumens). The catheter 52 includes at least two lumens—a main, or primary, lumen 66 and a secondary lumen 67. In some embodiments, the thrombectomy device (e.g., clot retriever) 58 is introduced through the main lumen 66 of the catheter 52. The thrombectomy device 58 may comprise an expandable member (e.g., a self-expanding stent or basket, an inflatable balloon-like member) positioned along an elongate shaft or at a distal terminus of an elongate shaft. At least a portion of the elongate shaft is flexible. In the illustrated embodiment, the distal end of the elongate shaft comprises an occlusive member 69 (e.g., a plug on the end of a shaft or wire that moves relative to the catheter 52) adapted to occlude the axial distal opening of the catheter 52 so that the particles (e.g., nanoparticles) can effectively form into agglomerated structures (e.g., stir bars or stir rods or spheroids) before leaving the catheter 52 into the blood vessel. However, other mechanisms or methods (e.g., expandable or inflatable members) may be used to occlude the axial distal opening of the catheter 52.

A rotating magnetic field and gradient can be applied (e.g., using an external magnet 33 that is a permanent magnet or an electromagnet) to facilitate formation of the agglomerates 20 as described elsewhere herein, and as schematically illustrated in FIG. 6B. The catheter 52 can be withdrawn proximally or the occlusive member 69 can be advanced distally or the occlusive member 69 can be transitioned to an unexpanded configuration in order to facilitate delivery of the agglomerated nanoparticles, or stir bars, 20 out of the catheter 52, as schematically illustrated in FIG. 6C. In some embodiments, the thrombectomy device 58 is a separate component from the wire or shaft having the occlusive member 69 and the two separate components are both delivered through the main lumen 66 of the catheter 52. Therapeutic, diagnostic, or theranostic materials (e.g., thrombolytic agents and/or contrast media) may also be delivered through the catheter 52 in conjunction with the agglomerated nanoparticles. In some embodiments, the elongate shaft of the thrombectomy device 58 comprises a central lumen through which the separate wire or shaft with the occlusive member 69 is introduced.

FIG. 6D schematically illustrates engagement of the thrombectomy device 58 with the clot 30 after retraction or withdrawal of the catheter 52. FIG. 6E schematically illustrates retraction or withdrawal of the thrombectomy device 58 with the captured clot 30. The previously-deployed nanoparticle agglomerates 20 and therapeutic, diagnostic, or theranostic materials (e.g., thrombolytic agents) 35 may advantageously act on any debris or clot fragments (e.g., emboli) 60 that flow downstream during the thrombectomy procedure or during the retraction or withdrawal of the thrombectomy device 58, thereby enhancing the safety and efficacy of the thrombectomy procedure. The nanoparticle agglomerates 20 and/or any accompanying diagnostic agents may also facilitate determination of the location of distal emboli utilizing an imaging modality, as described in more detail elsewhere herein.

FIG. 7 illustrates an embodiment of the catheter 52 (e.g., microcatheter) having multiple side openings or apertures 79 positioned along the distal end portion of the catheter 52. The openings 79 may branch off from a main lumen 66 or a secondary lumen 67 of the catheter 52. The openings 79 may advantageously facilitate delivery of nanoparticles 15 and/or therapeutic, diagnostic, or theranostic agents (e.g., thrombolytic agents such as tPA and/or plasminogen) 35 proximal to, within, and/or distal to the clot 30. Any number of openings 79 may be used and may have various shapes and sizes. The openings 79 may be positioned along the length of the clot 30 so as to facilitate lysis of the clot during a thrombectomy procedure when combined in use with a thrombectomy device delivered through the catheter 52. Thrombolytic agents may be held and trapped by the outer surface of the catheter 52 against the clot 30.

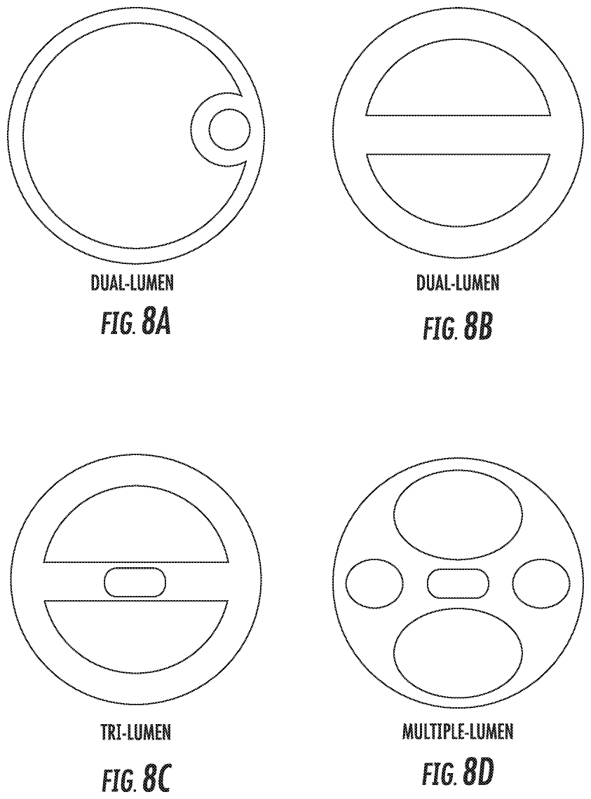

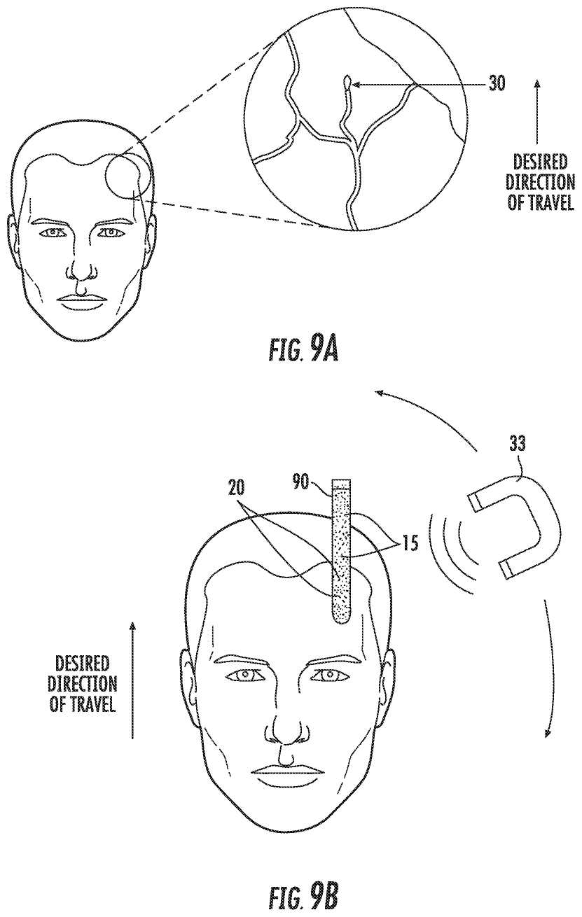

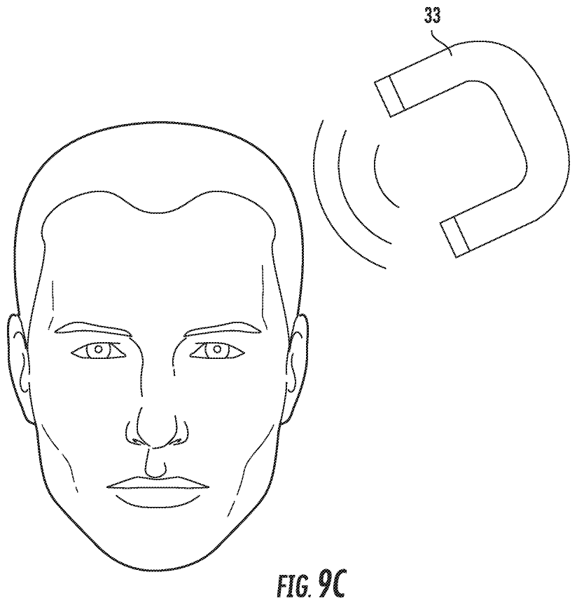

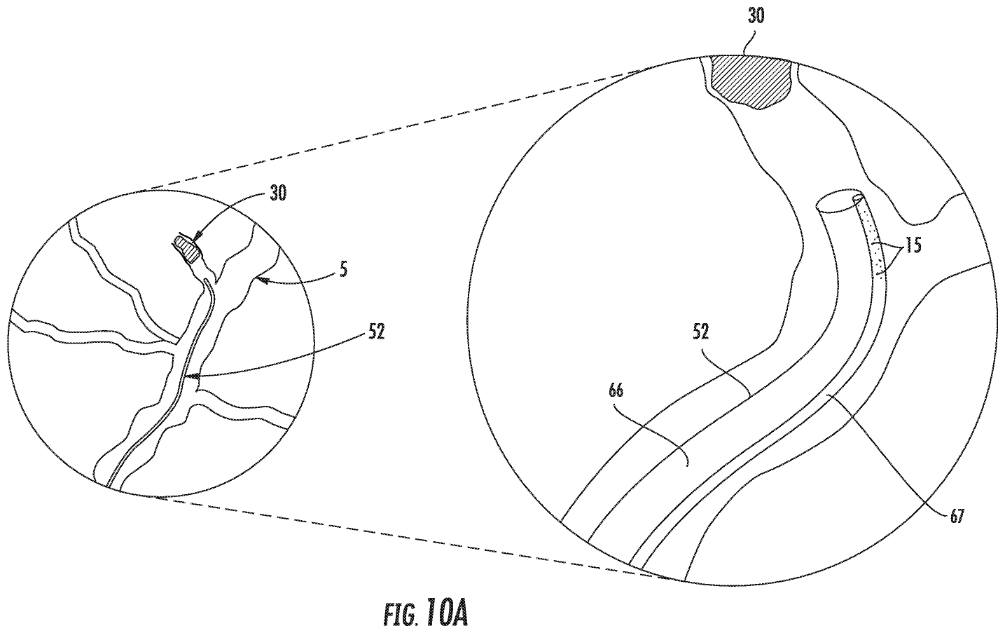

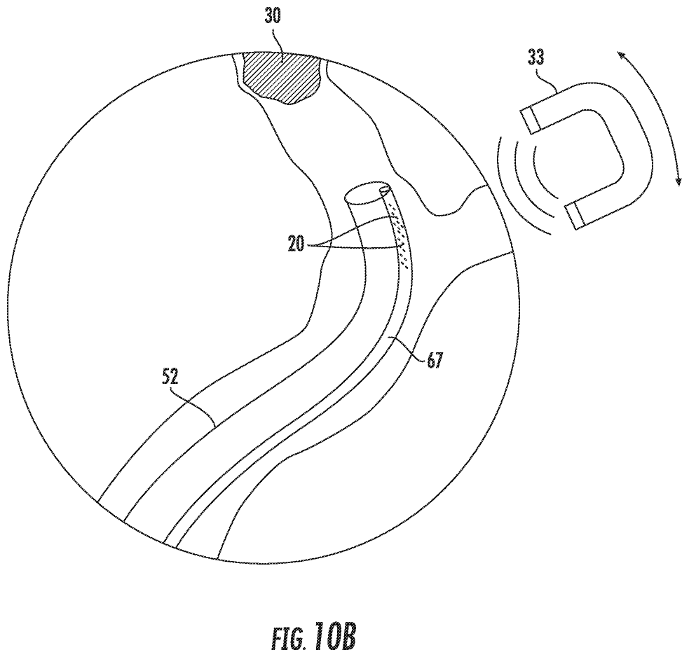

FIGS. 8A-8D illustrate cross-sectional views of various embodiments of the catheter 52 (e.g., microcatheter). FIGS. 8A and 8B illustrate cross-section views of two embodiments of a dual-lumen catheter. FIG. 8C illustrates a cross-section view of an embodiment of a tri-lumen catheter and FIG. 8D illustrates a cross-section view of an embodiment of a multiple-lumen catheter having five lumens. The catheter 52 may comprise one, two, three, four, five or more than five lumens as desired and/or required. Any of the lumens may be used to facilitate introduction of the thrombectomy device 58, nanoparticles 15 or the therapeutic, diagnostic, and/or theranostic agents 35. Introducers other than catheters may also be used.

### Control of Direction of Travel of Magnetic Particles

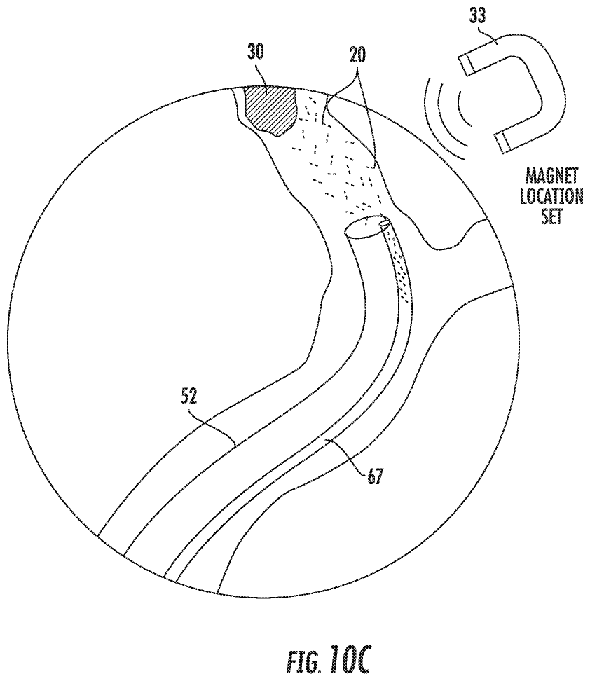

FIGS. 9A-9C and FIGS. 10A-10C schematically illustrate embodiments of methods to determine and effect delivery of the magnetic particles (e.g., nanoparticles) 15 along a desired direction of travel so as to increase the speed at which the magnetic nanoparticles 15 (and any accompanying therapeutic and/or diagnostic agents) arrive at a target location (e.g., clot location) and the number of magnetic nanoparticles 15 (and any accompanying therapeutic and/or diagnostic agents) arrive at the target location. Although FIGS. 9A-9C and FIGS. 10A-10C illustrate a clot as the therapeutic target and thrombolytic material as the therapeutic agent, other therapeutic targets can be substituted for the clot (e.g., a tumor, occlusion, obstruction) and other therapeutic agents may be substituted for the thrombolytic (e.g., trans-arterial chemotherapeutic agent, neuroprotectant, cardioprotectant).

With reference to FIG. 9A, a location of a clot 30 is identified using an imaging modality (e.g., CT angiogram) and a desired direction of travel of a thrombolytic is determined based on the identification of the location of the clot (e.g., based on imaging of the vasculature leading to the identified location of the clot). With reference to FIG. 9B, a tube or other container 90 containing multiple nanoparticles 15 can be held up at an external position adjacent to the identified location of the clot 30 and aligned with the desired direction of travel. The external magnet 33 can then be activated and the position and orientation of the external magnet 33 can be adjusted until an operator can visualize stir bar 20 formation and translation of the agglomerated particles (e.g., nanoparticles) in the tube or other container 90 in the desired direction of travel. Thus, the tube or other container 90 advantageously acts as a visual indicator to facilitate alignment of a desired rotation plane of the magnet or magnetic field to reach the location of the clot 30. With reference to FIG. 9C, after the adjustment is complete and the external magnet 33 has been appropriately positioned to effect the desired direction of travel, the magnet 33 can remain activated for a desired duration of time and nanoparticles and therapeutic and/or diagnostic agents may be delivered to effect desired therapeutic and/or diagnostic procedures (e.g., lysis of the clot 30).

With reference to FIGS. 10A-10C, the same control and adjustment of the positioning and orientation of the external magnet 33 can be accomplished through visualization of agglomerated particles (e.g., nanoparticles) (either themselves or contrast media attached to or otherwise co-located with the agglomerated nanoparticles) within the catheter 52 prior to delivery of the agglomerated nanoparticles (and any accompanying therapeutic and/or diagnostic agents) from the catheter 52. FIG. 10A schematically illustrates visualization of the nanoparticles 15 within the catheter 52 positioned adjacent to a location of a clot 30 within a blood vessel 5. Although not shown, the open distal end of the catheter 52 may be obstructed or occluded to prevent exit of the nanoparticles 15 at this stage. FIG. 10B schematically illustrates the formation of stir bars 20 after application of a rotating magnetic field and gradient using the external magnet 33. The position and/or orientation of the external magnet 33 can be adjusted until an operator can visualize stir bar formation and translation in the desired direction of travel utilizing an imaging modality (e.g., fluoroscopy, ultrasound, etc.). With reference to FIG. 10C, once the position and orientation of the external magnet 33 has been set, the secondary lumen of the catheter 52 can be opened (e.g., unoccluded) and the stir bars 20 of nanoparticles 15 (as well as accompanying thrombolytic) can be released from the catheter 52 while the rotating magnetic field and the magnetic gradient are applied by the external magnet 33 so that the stir bars 20 and accompanying thrombolytic can travel along the desired direction of travel toward the clot 30, thereby effecting lysis of the clot 30 in an efficient manner. Again, the “priming” of the stir bars 20 within the catheter 52 prior to release can advantageously result in less of the accompanying thrombolytic from being washed away downstream, thereby allowing for less thrombolytic to be used overall to adequately effect lysis of the clot, which results in increased efficacy and safety and decreased likelihood of adverse events caused by thrombolytics.

FIG. 11 schematically illustrates that the desired direction of travel may vary along a particular blood vessel in order to reach a location of a clot 30 or clot fragment (e.g., embolus) 60 due to the tortuous nature of the vasculature. As shown in FIG. 11, the desired direction of travel (and thus, adjustment of the position and/or orientation of the external magnet 33) may be varied multiple times in order to effect efficient travel (e.g., steering) to the location of the clot 30 or clot fragments 60. If the magnetic nanoparticles 15 are tuned or otherwise caused to be opaque to an imaging modality (e.g., through the properties of the nanoparticles themselves or through coating or conjugation or mixing with contrast media), the locations of the clot fragments 60 can be easily determined and treatment can be directed (e.g., steered) to the determined locations so that further ischemic stroke events (which may otherwise have been caused by a thrombectomy procedure) can be prevented or treated, thereby increasing the safety of the thrombectomy procedure and reducing the likelihood of adverse side effects or consequences of the thrombectomy procedure. In some embodiments, multiple permanent magnets may be positioned at different external locations and used to steer magnetic particles (along with accompanying therapeutic, diagnostic, or theranostic agent(s)) along a vessel or lumen or throughout a patient's body through multiple vessels or lumens. The one or more magnets may be used to control delivery of magnetic particles (along with accompanying therapeutic, diagnostic, or theranostic agent(s)) to multiple different locations by steering or guiding the delivery by adjusting orientations and positions of the magnets.

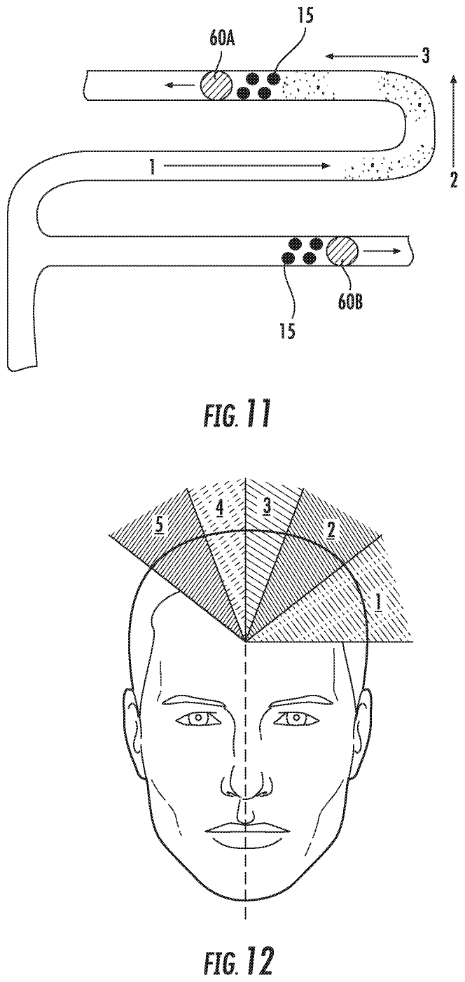

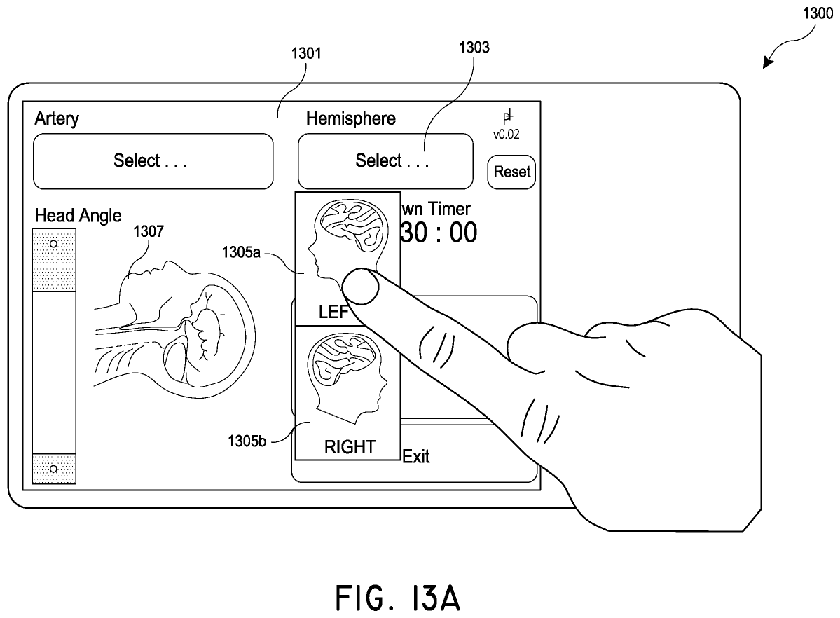

Also as shown in FIG. 11, clot fragments 60A,B may travel to various locations within the vasculature. By injecting particles (e.g., nanoparticles) 15 in conjunction with a thrombectomy procedure, the nanoparticles 15 may advantageously travel downstream along with clot fragments (e.g., emboli) 60 that break off from the clot 30 during the thrombectomy procedure and can bring thrombolytic and/or contrast media along with them to facilitate treatment and/or diagnostic procedures. For example, locations of the emboli 60 may be determined via imaging modalities (e.g., due to the opacity of the nanoparticles or due to the contrast agent that travels along with the nanoparticles) and additional nanoparticles 15 and thrombolytic 35 may be deployed to the determined locations. The locations of the emboli 60 may be determined by hot spots on the imaging caused by an increased presence of nanoparticles at a location caused by a downstream embolus 60 or multiple emboli. FIG. 12 schematically illustrates that various zones (e.g., Zones 1-5) of a subject's body may be identified and certain zones may be prioritized above other zones based on known locations of clots or clot fragments (e.g., emboli). The position and/or orientation of the external magnet 33 may be adjusted so as to effect a different desired direction of travel for each particular zone. For example, magnetic particles (and any diagnostic, therapeutic, or theranostic agents) may be steered towards different target locations. In some embodiments, multiple permanent magnets may be positioned at different external locations and used to steer magnetic particles (along with accompanying therapeutic, diagnostic, or theranostic agent(s)) along a vessel or lumen or throughout a patient's body through multiple vessels or lumens.

### Example Method of Operation and Magnetic Control System

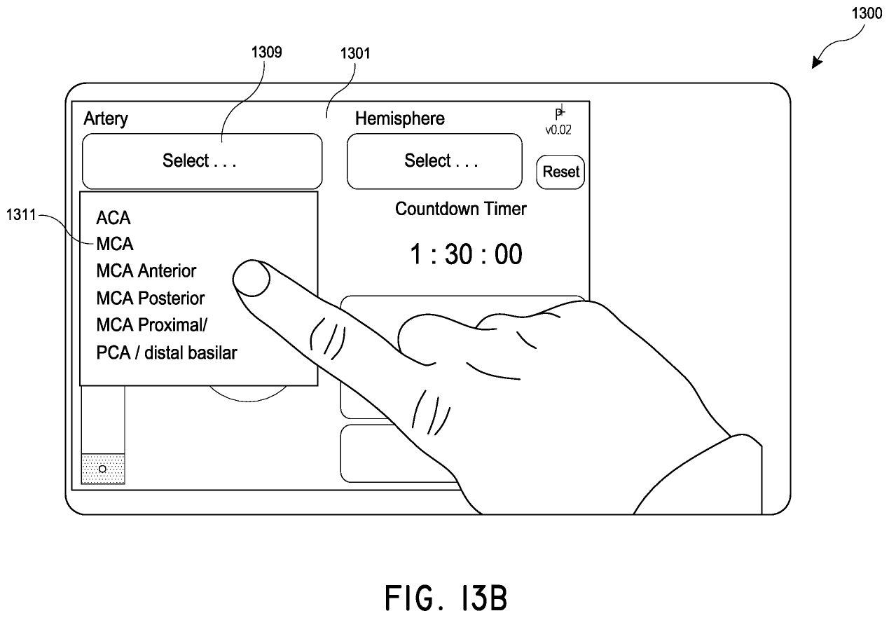

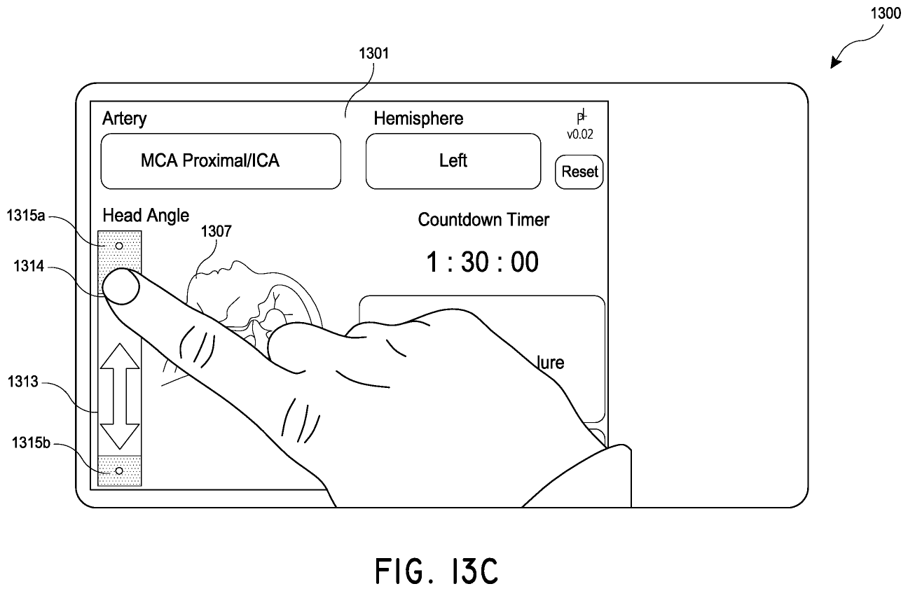

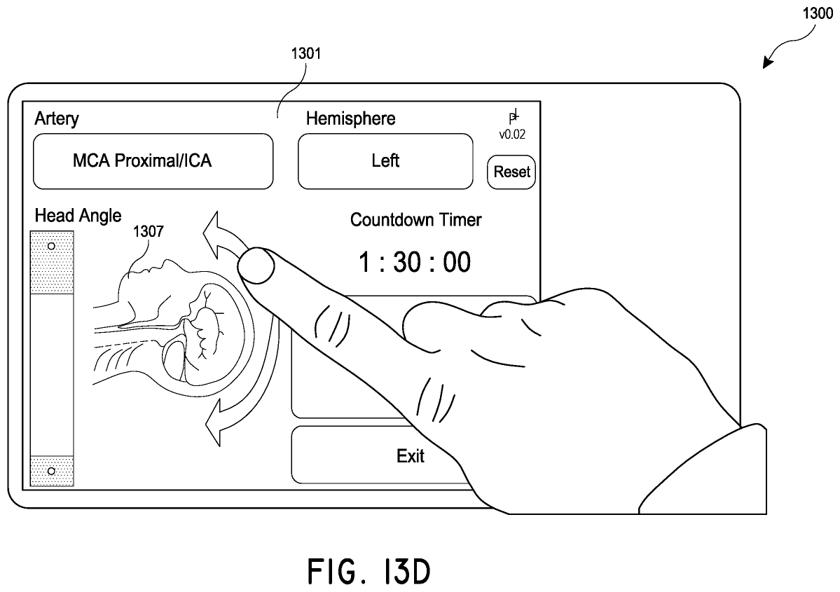

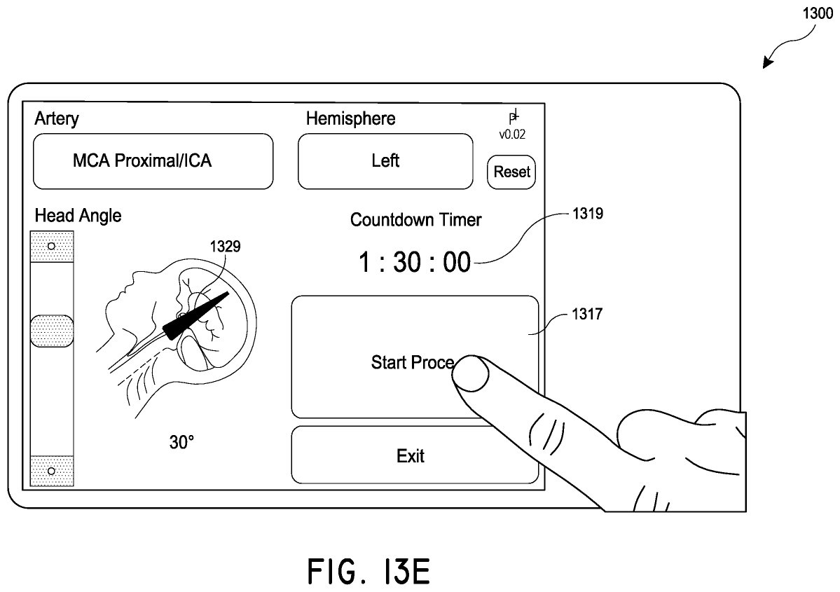

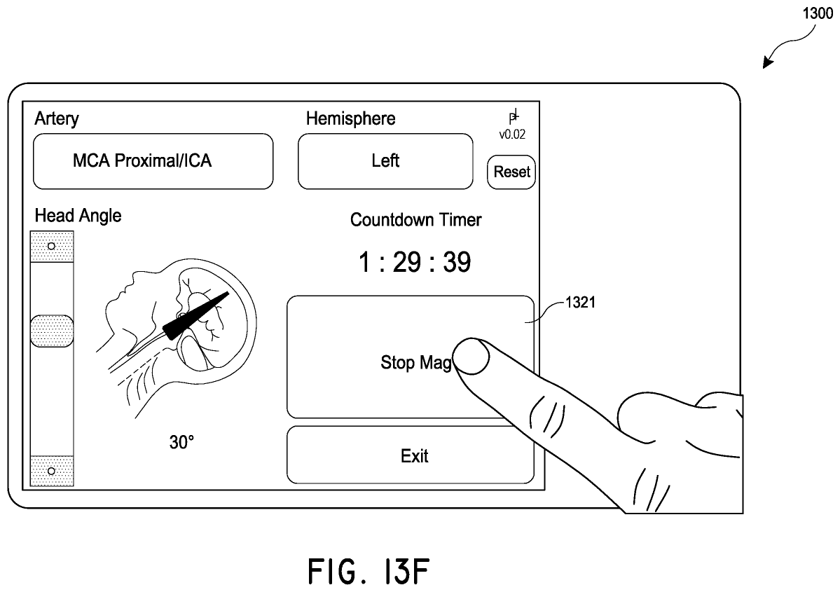

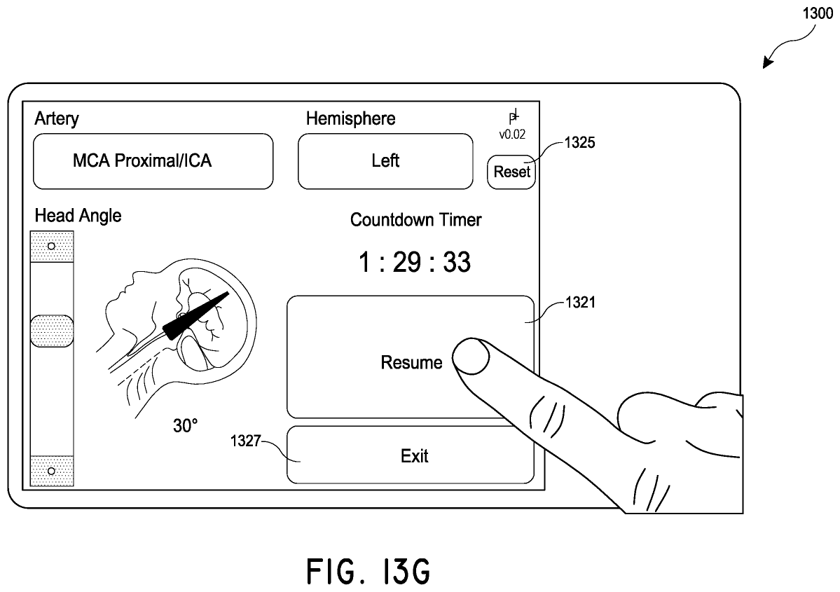

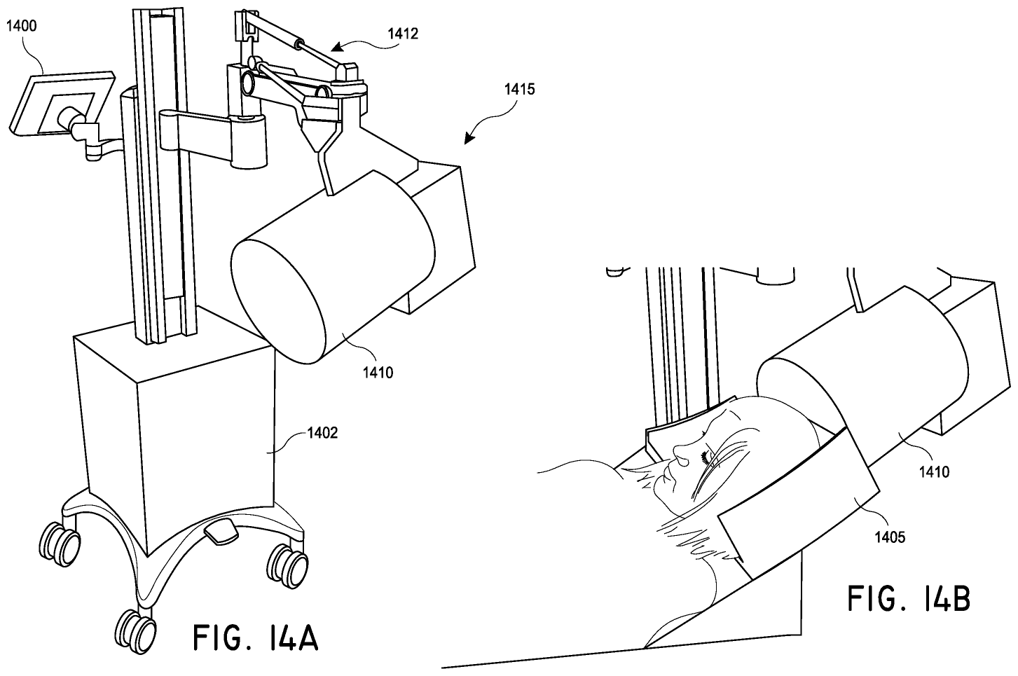

To illustrate an example method of operation, an example magnetomotive (e.g., magnetic control) system and user interface will be described with reference to FIGS. 13A-13G and FIGS. 14A and 14B, in accordance with embodiments of the invention. The magnetomotive system and associated magnetic particles can be configured, for example, to enhance infusion of co-administered agents into selected low flow vessels (e.g., where flow is less than or equal to about 1 cm/s) located within any body lumen. FIGS. 13A-13B illustrate an embodiment of a user interface module 1300 for use when operating the magnetomotive system 1415 to control magnetic rotors (e.g., magnetic nanoparticles) to deliver a therapeutic agent (e.g., tPA, plasminogen or other thrombolytic agent, neuroprotectant, cardioprotectant) to a therapeutic target (e.g., thrombus, clot or tumor) in a patient's head (e.g., vessel providing blood flow to the brain). The user interface module 1300 can be configured for use to treat or facilitate diagnostic imaging of other vessels or body structures of a patient as well.

The magnetomotive system 1415 can include a portable support base 1402 and an arm positioner 1412, as illustrated in FIG. 14A. The system 1415 can include a magnetic stator system configured to produce a desired magnetic field. For example, a magnetic stator system can include a neodymium-iron-boron permanent magnet block connected to a shaft and yoke assembly. In some embodiments, the yoke assembly is machined using carbon fiber plates to decrease weight and improve performance.

The permanent magnet block can be a single permanent magnet or multiple magnets. For example, the permanent magnet block can comprise two, three, four, six, eight, or some other number of NdBFe50 medium-temperature 2 inch cubes. A mechanical drive train can connect these assemblies to a pair of electric motors configured to vary in angulation and time to vary the magnetic field produced by the magnetic block. In some embodiments, the magnetic block can have a rotational frequency of at least about 1, 2 or 3 Hz and/or less than or equal to about 10 Hz (e.g., 2-4 Hz, 1-5 Hz, etc.) to produce a desired varying magnetic field. In some embodiments, the magnetic block is configured to produce a desired magnetic field at least about 6 inches from the surface of the magnetic block. In some embodiments, the magnetic block is configured to produce a magnetic field that is less than or equal to about 5 Gauss at about 54.6 cm inches from the magnetic block, and/or less than or equal to about 1 Gauss at about 94 cm from the block. In several embodiments, these mechanisms are housed in a protective cover that protects the operator and patient from mechanical hazards, as well as protects the elements and assemblies contained within the housing from hazards outside the housing.

The arm positioner 1412 can be configured to position and/or orient the magnetic stator system 1415 in a desired location, such as adjacent to a patient's head, during treatment, or into a stowed position when not in use. The system 1415 can include mechanisms to substantially secure the magnetic stator system in a desired location, such as locking or friction mechanisms. The system 1415 can advantageously include a touchscreen interface module 1300 configured to display information to the operator and receive input from the operator for use in controlling the system.

In one embodiment, the touchscreen interface module 1300 displays the user interface 1301 illustrated in FIGS. 13A-13G. The user interface module 1300 can aid in the proper operation of the system by allowing an operator to enter appropriate information, display progress of treatment, allow a user to manipulate the system, pause operation, modify operating parameters, and the like. For example, the user interface 1301 can provide the operator the ability to select a therapeutic target or region by cranial hemisphere and major arterial vessel (or vessel branch). The user interface 1301 can display information to the operator so the operator can verify the therapeutic target, a status of the magnetic stator system, and a time remaining for therapy delivery. In some embodiments, the therapeutic target or target location is identified without requiring imaging or visualization of the therapeutic target (e.g., without imaging of a clot). For example, the therapeutic target may be identified based on measured blood flow or other indications of fluid obstruction.

As an example of using the system and user interface, a treatment of a therapeutic target in the patient's head will be described with reference to FIGS. 13A-13G and FIGS. 14A and 14B. Although one embodiment of a magnetic control or stator system is referenced herein, the other magnetic control or stator systems described herein may also be used. The patient can be placed in a supine position with the patient's head positioned using a securing system, such as a head rest 1405, as shown for example, in FIG. 14B. The patient can be prepared to receive the treatment according to standard protocols of the treating institution. A magnet pod 1410 (which may include a rotatable permanent magnet and the mechanical mechanisms to effect rotation of the permanent magnet as described herein) of a magnetic control system 1415 can be positioned adjacent to a side of the patient's head proximate to an affected hemisphere, or the magnet pod 1410 can be positioned adjacent to the top or crown of the patient's head (as shown, for example, in FIG. 14B). In some embodiments, positioning the magnet pod 1410 of the magnetic control system 1415 adjacent to the side the head allows for imaging equipment to be placed adjacent a top of the patient's head. The headrest 1405 may not be used in some implementations.

Positioning the magnet pod or block 1410 of the magnetic control or stator system 1415 can include using one or more mechanical features, e.g., the positioning assembly 1412 (which may be composed of multiple independently controllable linkages or a single, unitary member) and portable support base 1402, to position and/or orient the magnetic stator system 1415 in a desired location relative to the patient. The positioning assembly 1412 may include multiple pivots, joints, and/or hydraulic mechanisms that each can be adjusted individually or in combination. The positioning assembly 1412 can adjust the magnet pod 1410 along multiple axes or without restriction (e.g., six degrees of freedom) in order to provide precise positioning with respect to a head angle of a patient. For example, the magnet pod 1410 may be configured to rotate about two or more axes of rotation. The positioning assembly 1412 may include locking mechanisms to prevent movement once a desired orientation and position is obtained. In some embodiments, the magnetic stator system 1415 can be positioned perpendicular to the patient's head and level with the patient's ear at a distance of between 2 and 20 cm (e.g., between 2 and 6 cm, between 4 and 10 cm, between 6 and 12 cm, between 8 and 20 cm, overlapping ranges thereof, 8 cm, or any distance within the recited ranges) from the patient's head. As another example, the magnetic stator system 1415 can be positioned parallel with the patient's head and adjacent the top or crown of the patient's head, as shown for example in FIG. 14B. The magnetic stator system 1415 can be configured to be substantially secured in place during use or it can be configured to move during use through manual operation, automatic operation, or some combination thereof. In some embodiments, the head rest 1405 can be used to position the patient's head and to substantially secure it in a fixed location and/or orientation. The head rest 1405 can be disposable. In one embodiment, the head rest 1405 has one side cut shorter than the other to facilitate alignment of the magnetic stator system 1415 with the patient.

The operator can select an affected hemisphere of the head or brain which contains the therapeutic target (e.g., clot or other fluid blockage). In FIG. 13A, the operator can use the interface 1301 to select the affected hemisphere through a hemisphere selection element 1303 where the selection corresponds to a side of the head where therapy is to be delivered. In some embodiments, the user interface 1301 presents two options in the hemisphere selection element 1303, a “LEFT” and a “RIGHT” option, with associated images 1305a and 1305b. In response to the operator's selection, the head angle image 1307 can change to reflect the selection.

In some embodiments, the operator selects a specific arterial branch to target. In FIG. 13B, the operator can use the interface 1301 to select a targeted artery through an artery selection element 1309. The artery selection element 1309 can include a list of arterial branches and the operator can select the specific arterial branch to target. By touching the artery selection element 1309, a list of available selections 1311 can be displayed. For example, the list of available selections 1311 illustrated includes ACA (anterior cerebral artery), MCA (middle cerebral artery), MCA Anterior, MCA Posterior, MCA Proximal, ICA (proximal MCA/internal carotid), PCA/distal basilar (posterior cerebral artery/distal basilar). Upon selection, the image associated with the head angle 1307 can show an arrow 1329 (shown in FIG. 13E) predicting a direction of infusion of the therapeutic agent.

In several embodiments, the operator sets or inputs a head angle of the patient, where the head angle is the angle of the patient's head relative to horizontal. The head angle of the patient can be changed during treatment using the user interface 1301. In FIGS. 13C and 13D, the operator can use the interface 1301 to set the head angle of the patient using a bar slider 1313, as illustrated in FIG. 13C, or by manipulating the head angle image 1307, as illustrated in FIG. 13D. Dragging the slider 1314 changes the head angle of the patient from 0 degrees to 90 degrees, where 90 degrees corresponds to when the patient is sitting up. Tapping above or below a current position of the slider 1314 can increase or decrease the head angle value by a defined amount (e.g., 5 degrees, 10 degrees, 20 degrees). Tapping upper and lower boxes 1315a and 1315b, respectively, can increase or decrease the head angle by 1 degree. As illustrated in FIG. 13D, the operator can drag a finger or input device on the interface 1301 around the head angle image 1307 to adjust the head angle.

In accordance with several embodiments, once the affected (e.g., target, hemisphere, targeted artery, and head angle are set, the operator can begin the procedure. FIG. 13E illustrates a “Start Procedure” button 1317. Before the affected hemisphere, targeted artery, and head angle are set, the button 1317 can be disabled and can indicate that it is disabled through the use of color, text, or other indicator. In some embodiments, the button 1317 does not appear until the parameter inputs are entered. For example, the button 1317 can be grey before the selections above have been made and green after. By pressing the “Start Procedure” button 1317, the magnetic stator system can be activated, at which point particle infusion, or introduction, should begin.

In one embodiment, the duration of infusion is about 60 minutes. In various embodiments, the duration of infusion is between 5 minutes and 120 minutes (e.g., between 5 minutes and 20 minutes, between 10 minutes and 30 minutes, between 15 minutes and 45 minutes, between 30 minutes and 60 minutes, between 45 minutes and 90 minutes, between 60 minutes and 120 minutes, overlapping ranges thereof, or any time duration within the recited ranges. In some embodiments, the system automatically suspends after a predetermined time (e.g., 90 minutes) has elapsed with the magnetic stator system in an activated state, as indicated by the countdown timer 1319. In accordance with several embodiments, the magnetic stator system continues to operate for a certain time period (e.g., about 30 minutes) after infusion is complete. As illustrated in FIG. 13F, when the operator presses the “Start Procedure” button 1317, it can be changed to or replaced by a “Stop Magnet” button 1321. By pressing the “Stop Magnet” button 1321, the magnetic stator system can be deactivated, the countdown timer 1319 can be paused, and the button 1321 can change to or be replaced by a “Resume” button 1323, as illustrated in FIG. 13G. When the magnet has been stopped, the operator can adjust the head angle of the patient, reset the system by pressing the reset button 1325, and/or exit the system by touching the exit button 1327. Pressing the “Resume” button 1323 can change it back to the “Stop Magnet” button 1321, in accordance with one embodiment.

### Compositions for Use in the System

Various formulations of magnetic nanoparticles, whether formulated in combination with pharmaceutical compositions or not, may be used for administration to a patient. Those of skill in the art will recognize how to formulate various therapeutic agents (e.g., pharmaceutical compositions, drugs and compounds, neuroprotectants, cardioprotectants) for co-administration with the magnetic nanoparticles hereof, or administration separate from the nanoparticles. In some embodiments, various formulations of the magnetic nanoparticles thereof may be administered neat (e.g., pure, unmixed, or undiluted). In some embodiments, various formulations and a pharmaceutically acceptable carrier can be administered, and may be in various formulations. For example, a pharmaceutically acceptable carrier can give form or consistency, or act as a diluent. Suitable excipients include but are not limited to stabilizing agents, wetting and emulsifying agents, salts for varying osmolarity, encapsulating agents, buffers, and skin penetration enhancers.

Some embodiments of magnetic nanoparticles can be formulated with a coating that is non-specialized toward any particular therapeutic agent (e.g., chemical or pharmaceutical composition) or diagnostic agent. In some embodiments, the coating could absorb or adsorb a wide variety of therapeutic, diagnostic and/or theranostic agents. The coating can be formulated to be thin, as well, so as to not substantially interfere with the mutual interaction of the magnetic nanoparticles. As a result, chemicals can be delivered in a more efficient way, reducing the dose, cost, and dose-dependent side effects.

Magnetic nanoparticles with the non-specialized coating could be acquired and mixed with an operator-selected agent prior to injection into a subject. Thus, the operator can select a suitable agent for the particular application. The devices used to attach a therapeutic agent (e.g., a chemical, a thrombolytic drug, plasminogen, neuroprotectant, cardioprotectant) to the magnetic nanoparticles can include a traditional vortex mixer, ultrasonic mixer, a device which manipulates a magnetic field to result in dispersing the particles and mixing them with the chosen therapeutic agent, or other suitable mixing device. Operator-selected agents can include, for example, coatings which enhance the behavior of the nanoparticles in blood by making them either hydrophilic or hydrophobic; coatings which buffer the nanoparticles and which optimize the magnetic interaction and behavior of the magnetic nanoparticles; contrast agent or agents which allow visualization with magnetic resonance imaging, X-ray, Positron Emission Tomography (PET), or ultrasound technologies; drugs which accelerate destruction of a circulatory system blockage; tissue plasminogen activators (tPA), plasminogen, streptokinase, urokinase, recombinant tissue plasminogen activators (rtPA), alteplase, reteplase, tenecteplase, other drugs, or any combination of these. Coating magnetic nanoparticles with a non-specialized coating can have advantages. For example, magnetic nanoparticles can be injected without tPA or other such drugs attached, but the nanoparticles can be used to concentrate drugs which are introduced separately through the use of fluid currents. In this way, the drugs can be more effective due to the ability of the nanoparticles to concentrate the dose at the targeted location or therapeutic target. In some embodiments, such a process can reduce the effective dosage required by two or three orders of magnitude, saving in cost and possible deleterious drug side-effects. In some embodiments, the magnetic nanoparticles can include a specialized coating to facilitate bonding or attachment of a particular therapeutic agent.

The magnetic nanoparticles can be biologically inert and not able to be metabolized. In some embodiments, the coating comprises a polyethylene glycol (PEG) coating, a polylactic acid (PLA) coating, a polyvinyl acetate (PVA) coating, a dextran coating, and/or an oleic acid coating. The coating can advantageously remove a charge of the magnetic nanoparticle and prevent or reduce the likelihood of hemolysis. The coating can serve as a platform or scaffolding for attachment of therapeutic agents. In some embodiments, the coating includes an intermediate coating (e.g., an acid coating) to facilitate attachment of a therapeutic agent. In some embodiments, the coating can be modified to include an acid layer to facilitate conjugation or bonding of therapeutic agents to the coating. The coating may be layered in some embodiments, and optionally provide controlled time release. The magnetic particles (e.g., nanoparticles described herein) may be embedded within a polymer matrix.

In some embodiments, the magnetic nanoparticles described herein can be used in purifying biomolecules through magnetic bioseparation. For example, by oscillating a magnetic field, magnetic nanoparticles can enhance a process of purifying biomolecules. In some embodiments, the surface of the magnetic nanoparticles can be functionalized to attach desired molecules. For example, diagnostic markers for various diseases such as cancer can be attached to the magnetic nanoparticles. The magnetic nanoparticles can be used to enhance magnetic resonance imaging or other contrast agents to improve MRI or other imaging results. In several embodiments, the magnetic nanoparticles can be used as vectors for drug delivery by attaching drugs to the surface of the particles and directing them to a site. In accordance with several embodiments, the magnetic nanoparticles can be directed to a site to deliver a critical concentration of material with a therapeutic agent attached to the nanoparticles to the site. In some embodiments, the diagnostic markers, contrast agents or drugs are not attached to the nanoparticles but simply mixed with or co-administered with the nanoparticles.

### Administration of Magnetic Nanoparticles

Magnetic particles (e.g., nanoparticles) and tPA (or other agent) can be infused concurrently through systemic intravenous infusion or other infusion method described herein (e.g., intra-arterially through a microcatheter) or in WIPO Publication No. 2013/173235 (e.g., infusion methods that facilitate mixing of the particles so as to avoid settling during infusion). In embodiments employing systemic infusion, multiple highly-concentrated boluses of magnetic nanoparticles may be infused over time. Using a control system such as the control systems described herein, an operator can use the magnetic stator system to magnetically manipulate the magnetic nanoparticles to form magnetic rotors which act to create or increase blood flow currents directing the tPA to the clot. In this way, the magnetic rotors can increase tPA diffusion and accelerate clot destruction. The operator can control the magnetic stator system to create a desired varying magnetic field.

By alternating a rotational direction of the magnetic stator system, the operator can direct the magnetic rotors within a vessel. For example, within a vessel, a velocity of blood increases with distance from the vessel wall, where the velocity is approximately zero. A clotted vessel branch will obstruct fluid flow resulting in the velocity dropping to zero at the opening of the branch. Within such low velocity regions, magnetic nanoparticles generally assemble to be controlled by the magnetic stator system. When assembled, the magnetic stator system can agglomerate the magnetic nanoparticles into larger structures (e.g., magnetic rotors having an oblong shape). With a varying magnetic field, the magnetic rotors can rotate, resulting in an end-over-end motion that results in the magnetic rotors traveling into or next to the blocked branches. The resulting rotational motion of the magnetic rotors can create new currents or increase low-velocity currents. The resulting currents can concentrate a therapeutic agent in an otherwise inaccessible or difficult to access region. By changing the rotation of the magnetic stator system, additional branches can be infused. For example, different rotational directions can result in the magnetic rotors traveling to different branches. Rotational directions can be alternated to direct magnetic rotors to multiple branches (e.g., steering). In accordance with several embodiments, the magnetic rotors need not contact the therapeutic target to treat (e.g., reduce, erode, lyse, degrade, clear, or otherwise address) the target. For example, the magnetic rotors can facilitate treatment (e.g., removal, lysis or erosion) of a thrombus or clot without scraping or contacting the clot, or without the contact being the primary cause of action.

In addition, in one embodiment, when the magnetic rotors comprise magnetic nanoparticles, such as magnetite or another ferromagnetic mineral or iron oxide, the rotors can be manipulated in a way that improves mixing of a chemical or pharmaceutical agent that is in the vicinity of the magnetic nanoparticles. The use of the magnetic gradient combined with a time-varying magnetic field allows for flow patterns to be created which then amplifies the interaction of the chemical or pharmaceutical. This mechanism has been observed in animal models for the destruction of clots within the endovascular system using tPA as a thrombolytic. The pharmaceutical compositions can also be attached to the magnetic nanoparticles to perform the same function. As a result, less of those agents may be used for patient treatment provided that the nanoparticles are able to be navigated to and interact with the desired targets using the magnetic gradient and the time-varying magnetic field of the system.

In accordance with several embodiments, systems and methods are provided for delivering non-dispersible or difficult to disperse agents (such as embodiments of the magnetic nanoparticles described herein). Administering magnetic nanoparticles by injection can present challenges where a substantially consistent infusion mass is desired as a function of time. The infusion mechanism can include syringes, drip bags, reservoirs, tubing, drip chambers, other mechanisms, or any combination of these. Magnetic particles can be dispersed in solutions such as, for example, saline, Ringer's solution, dextrose solution, and the like. After a certain amount of time has elapsed in such solutions, magnetic particles can settle near the bottom of the solution due primarily to gravitational forces on the particles possibly resulting in an inconsistent infusion mass.

For example, in certain applications, the magnetic nanoparticles are supplied in a single-dose vial containing about 500 mg of magnetic nanoparticles dispersed in about 17 mL of phosphate buffered saline (PBS), and are designed to be infused over the course of about an hour. These magnetic nanoparticles can settle out of dispersion in about 5 to 10 minutes. Thus, the magnetic nanoparticles would settle faster than the time used to administer them, thereby causing the infusion mass to be inconsistent.

Some embodiments of the magnetic nanoparticles described herein are non-dispersible or difficult to disperse in a fluid. Some embodiments of the magnetic nanoparticles described herein include a magnetically-strong, relatively large, single-crystalline core having a diameter greater than or equal to about 50 nm and/or less than or equal to about 200 nm. The magnetic nanoparticles can also be coated with a relatively thin polyethylene glycol coating (e.g., less than or equal to about 5 nm, 10 nm, 20 nm, etc.) to reduce the charge associated with the particles. To disperse such nanoparticles in a fluid, e.g. saline, the thickness of the nanoparticle coating can be substantially increased and/or the viscosity of the dispersion medium can be increased. In some embodiments, systems and methods are provided for maintaining a substantially consistent infusion mass without altering the thickness of the nanoparticle coating or the viscosity of the dispersion medium.

In some embodiments, magnetic particles are made more dispersible by coating the particles with a relatively thick coating. As an example, a relatively thick coating can be applied to magnetic nanoparticles to ensure the nanoparticles remain in steric repulsion, such as magnetite or hematite nanoparticles coated with Dextran or polyethylene glycol surrounding a relatively small polycrystalline, magnetic core (e.g., the magnetic core has a diameter less than or equal to about 20 nm). In some embodiments, systems and methods for maintaining a consistent infusion mass can infuse magnetically strong particles without a relatively thick coating, e.g., magnetic nanoparticles described herein having a single-crystalline core with a diameter greater than or equal to about 20 nm and/or less than or equal to about 200 nm.

Typically, magnetic particles experience steric repulsion because they have a relatively thick coating such that they remain substantially dispersed throughout the infusion process. In some applications, magnetic particles are coated with a coating to reduce the magnetic susceptibility of the particles, like when the magnetic particles are used as contrast agents for use in magnetic resonance imaging. In some applications, magnetic particles are coated with biodegradable substances, hydrophobic drugs, or other such coatings. Such coatings can be effective in increasing the dispersion of the particles in a solution. In some embodiments, the magnetic nanoparticles described herein and the infusion methods and systems described herein advantageously allow the magnetic nanoparticles to substantially remain in dispersion throughout an infusion process without experiencing steric repulsion and/or without requiring a relatively thick coating.

Delivery of Diagnostic, Therapeutic or Theranostic Agents in Conjunction with Magnetic Particles

In addition to facilitating delivery of thrombolytics or other therapeutic agents to fluid obstructions, the magnetic control systems and nanoparticles described herein may advantageously be used to deliver contrast media, drugs or other materials to various locations within the body that may be difficult to access (e.g., due to location, small size, obstructions, etc.). In a typical flow situation, there are locations where the flow does not effectively penetrate or target the intended site. The formation and navigation of the stir bars of nanoparticles may facilitate delivery of contrast media, drugs or other materials to locations that could not be accessed, or could not easily be accessed, without use of the stir bars. For example, the magnetic control systems can be used to move nanoparticles within small channels in a manner superior to approaches attempted with non-varying magnetic fields. The combined use of the magnetic gradient with a time-varying magnetic field allows for the nanoparticles to travel into small vessels, at which point therapy can be directed. In some embodiments, magnetic control systems and nanoparticles described herein may facilitate diagnostic or combined therapeutic and diagnostic (e.g., theranostic) procedures. As one example, contrast media (e.g., contrast agents) and/or drugs could be conveyed to a tumor and the tumor could be imaged, treated or both imaged and treated (simultaneously or sequentially). Theranostic agents may include any agents configured to simultaneously facilitate both therapy and diagnosis (e.g., radioiodine, biologics, iron oxide nanoparticles, quantum dots, carbon nanotubes, gold nanoparticles, and silica nanoparticles).

In conventional imaging procedures, the images must be taken extremely quickly and large quantities of contrast media must be delivered in order to adequately capture images because the contrast media is swept away by normal blood flow or gravity or other causes. Some procedures may involve deployment of balloons or other occlusive members to stop blood flow to facilitate imaging. The delivery of too much contrast media can result in kidney failure and occlusion of flow can also result in adverse consequences. By magnetically controlling the stir bars, an operator can advantageously keep contrast media and/or drugs in a location for an extended period of time to facilitate imaging and/or therapeutic treatment, while reducing the required quantity of contrast media and/or therapeutic agents (e.g., drugs) and without having to use occlusive members to occlude flow. For example, while ordinary blood flow would cause contrast media and/or drugs to flow downstream of a therapeutic and/or diagnostic target quickly, the formation and control of stir bars may counteract the normal blood flow and cause the contrast media and/or drugs to remain in place adjacent or in contact with the therapeutic and/or diagnostic target (e.g., a tumor). In some embodiments, drugs can be mixed with contrast media to get to a target treatment location (e.g., tumor location) or to be held in the target location for treatment.

Contrast media can be any substance used to enhance the contrast of structures or fluids within the body in medical imaging. The contrast media can include, for example, contrast agents, iodinated contrast media, ionic iodinated contrast media, lymphatic staining agents, magnetic resonance imaging contrast media, miscellaneous diagnostic dyes, non-iodinated contrast media, non-ionic iodinated contrast media, ultrasound contrast media, iodine, barium, gadolinium, ethiodoized oil, gadoterate meglumine, iodixanol, iohexol, microbubble contrast agents, radiopharmaceuticals, and/or any other contrast media. The contrast media may be delivered directly or locally to a target location through a catheter such as described herein, through systemic intravenous introduction, through inhalation to the lung, by injection into muscle or skin or underneath the skin, nasally, rectally, vaginally, orally, through inhalation to the lung, and by injection into muscle or skin or underneath the skin. The contrast media may be delivered together with the magnetic nanoparticles or separately.

In accordance with several embodiments, contrast media, bioluminescence or other materials may be attached to (e.g., conjugated to or adsorbed to) or doped into the nanoparticles for chemical, magnetic, therapeutic, diagnostic, theranostic and/or imaging reasons. Example contrast coatings include contrast coatings detectable by x-ray, PET, MR and ultrasound imaging technologies. Such coatings can advantageously provide the ability to reconstruct a vessel which would normally be invisible due to the lack of blood flow in that region Likewise, the ability to control and recollect the magnetic nanoparticles can result in less toxic side effects, which may result from use of traditional contrast agents. For example, X-ray contrast agents typically require multiple injections because they are swept away with blood flow and are not able to travel in high concentrations down low-flow vessels.

In some embodiments, the magnetic nanoparticles (e.g., monocrystalline or polycrystalline iron oxide nanoparticles) themselves constitute contrast agents based on the makeup of the nanoparticles and can be opaque to certain imaging modalities or technologies. In various embodiments, the nanoparticles may comprise at least one of gadolinium, manganese, copper, nickel, cobalt, zinc, germanium, gold, silver, compounds comprising group II (A or B) and group VI elements, compounds comprising group IV and group VI elements, bioluminescence agents, combinations thereof, and the like. In some embodiments, the imaging technologies can be appropriately tuned so as to facilitate imaging of the nanoparticles or stir bars formed from the nanoparticles. In some embodiments, ultrasound-based diagnostic imaging or detection is based on Doppler imaging or detection which provides information about fluid flow. For example, diagnostic ultrasound (e.g., Doppler ultrasound) can be tuned to the rotational frequency of the rotating magnetic nanoparticle agglomerates or stir bars (e.g., 3 Hz). The feedback may be used to confirm location of the magnetic nanoparticles or to confirm recanalization has occurred. An ultrasound imaging system can image using frequencies from 1 and 18 MHz. The ultrasound images generated by the ultrasound-based imaging system can be two-dimensional, three-dimensional, or four-dimensional images. Tuning can also be effected utilizing the other imaging modalities described herein. For example, magnetic resonance imaging machines can be appropriately tuned so that the nanoparticles act as MRI contrast agents.

The diagnostic or imaging modalities or technologies may include X-ray, ultrasound, radiography, magnetic resonance, nuclear medicine, photo acoustic, thermography, tomography (PET, CT), and/or any other modalities or technologies. These imaging technologies can be used to visualize the magnetic particles (for example, if the magnetic particles are coated with a coating or conjugated to a substance or compound that facilitates visualization or imaging) or tissue surrounding or adjacent contrast media. For example, transcranial ultrasound imaging could be used to confirm clot destruction visually in a cranial embolism or stroke. The imaging technologies can be transmit images to a display device to provide an operator real-time feedback so that the operator can navigate or otherwise control movement of the magnetic nanoparticles.

The magnetic control systems and magnetic nanoparticles may be used in conjunction with any diagnostic or imaging scan, such as but not limited to angiograms, arteriograms, venograms, PET scans, CT scans, X-rays, portograms, elastography scans, lymhography scans, thermograms, sonograms, encephalograms, and/or the like. The contrast media and/or magnetic nanoparticles may be delivered to or through any body lumen, channel, space, volume or passage, including vasculature, Fallopian tubes, cerebrospinal spaces or passages, gastrointestianal tract (e.g., intestines, colon), ureters, lymphatic system (lymph nodes), intraosseous locations (e.g., bone cavities or spaces), liver, lungs, heart, pericardium, thoracic cavity, brain, etc. Other target locations or agents described in WIPO Publication No. 2013/173235 may also be used.

The magnetic control systems and magnetic nanoparticles described herein may also be used to facilitate identification, and clearing, of obstructions within in-dwelling catheters (e.g., in conjunction with cathograms). For example, contrast media may be delivered in conjunction with magnetic nanoparticles to facilitate imaging of flow within the in-dwelling catheters to determine the location of the obstructions or blockages within the catheters and then to facilitate clearing of the obstructions or blockages without having to remove and/or replace the catheters.

### Imaging and Real-Time Feedback

In one embodiment, the magnetomotive or magnetic control systems (such as those described herein) can make use of an easy-to-understand user-interface which allows the user to control the rotation plane of the magnetic field in a way that is not presently found. In some embodiments, the user interface comprises a touchscreen display. Furthermore, imaging technologies can be incorporated into or used in combination with the user interface such that an operator can have real-time feedback of the position of the magnetic particles (e.g., nanoparticles), allowing for dynamic control and navigation (e.g., steering). This can aid the operator to take steps to increase the effectiveness of the process, for example, by introducing more nanoparticles or more chemical agents. Images of the patient and/or regions of interest can be incorporated into a user face to aid an operator, physician, technician, or the like to plan a navigation route for the magnetic nanoparticles. Planning a navigation route can comprise identifying a therapeutic target, such as a clot, choosing a practical injection site for the nanoparticles, and planning a route through the patient's vasculature to arrive at the targeted object. During the actual navigation of the magnetic nanoparticles, the operator can use the original images used to plan the navigation or the user interface can receive updated images to show the operator, thus providing real-time imaging and feedback to the operator. The real-time user-interface can be coupled with a single-axis or multi-axis robotic arm to allow the operator to substantially continuously control the direction of nanoparticle infusion in real-time.

As an example, the real-time user interface can incorporate image information from an imaging system. The imaging system can be a system incorporating one or more imaging modalities, configured to provide imaging data to the magnetomotive system. The imaging data can be derived from x-ray data, PET data, MR data, CT scan data, ultrasonic imaging data, or other imaging modality data. In some embodiments, the magnetic nanoparticles themselves act as contrast agents in conjunction with an imaging modality.

The magnetomotive system, in one embodiment, receives imaging data from the imaging system. In some embodiments, the imaging data comprises information derived from an imaging modality that, in use, provides information about vasculature of a subject, relative position of magnetic nanoparticles, fluid flow, fluid obstructions, or any combination of these. For example, the imaging system can produce image data based on ultrasound-based imaging. The imaging system can transmit sound waves aimed at an area of interest and interpret the echoed waves to produce an image. The ultrasound-based imaging system can be configured to provide imaging data in real-time and can be configured to identify fluid flow, tissue, liquid, magnetic nanoparticles, and the like. In some embodiments, ultrasound-based imaging is based on Doppler imaging which provides information about fluid flow. The ultrasound imaging system can image using frequencies from 1 and 18 MHz. The ultrasound images generated by the ultrasound-based imaging system are two-dimensional, three-dimensional, or four-dimensional images.

The magnetomotive system, in one embodiment, registers a reference frame of the magnetomotive system to a reference frame of the imaging system such that the imaging data from the imaging system is mapped to positions relative to the magnetomotive system. In some embodiments, registering the reference frames includes identifying elements of a received image and mapping those elements to positions within a subject. In some embodiments, registering the reference frames includes receiving information about the image system itself such as a physical orientation of an imaging device relative to a subject, depth of scan or image, field of view, and the like such that the magnetomotive system can map the received image relative to a coordinate system of the magnetic system. For example, an ultrasonic imaging system can send information to the magnetomotive system about the frequencies transmitted into the targeted area, the orientation of the imaging system relative to the subject, the position of the imaging system relative to the patient, or any combination of these. As another example, a CT system can include information about the depth of scan of an image, the field of view, the orientation of the system relative to the patient, and the like.

In one embodiment, the magnetomotive (e.g., magnetic control) system identifies the magnetic nanoparticles within the imaging data received from the imaging system to track the particles, to navigate the particles, to switch between control modes (e.g. collection mode, vortexing mode, navigation mode, etc.), to monitor drug diffusion, or any combination of these. Identifying the magnetic nanoparticles can include analyzing the imaging data for signals associated with magnetic nanoparticles. For example, in ultrasonic imaging the magnetic nanoparticles can have a distinctive signal in an image due to their motion, composition, position, behavior, orientation, or any combination of these. As another example, in PET systems the magnetic nanoparticles can have a distinctive and/or identifiable signal in an image based on attached contrast agents, the density or composition of the nanoparticles, the position of the nanoparticles, or the like.

The magnetomotive (e.g., magnetic control) system can determine a position of the magnetic nanoparticles relative to the magnetomotive system, based on the registration of the reference frames. The magnetomotive system can plan a navigation path from the identified position of the magnetic nanoparticles to a desired location within the subject based on the imaging data from the imaging system. For example, the navigation path can include an acceptable path through the vasculature of the subject from the current location of the magnetic nanoparticles to the targeted structure, such as an occlusion. In some embodiments, planning a navigation path comprises identifying a therapeutic target, such as a clot, choosing a practical injection site for the nanoparticles, and planning a route through the patient's vasculature to arrive at the therapeutic target.

The magnetomotive system can manipulate a magnetic field produced by the magnetic system to navigate the magnetic nanoparticles according to the navigation path. In some embodiments, manipulation of the magnetic field causes the magnetic nanoparticles present within the vasculature to agglomerate into a plurality of magnetic nanoparticle stir rods and causes the agglomerated structures to travel through fluid within the vasculature by repeatedly walking end over end away from the magnetic field in response to rotation of the magnetic nanoparticle rods and the magnetic gradient and (b) flowing back through the fluid towards the magnetic field in response to the rotation of the magnetic nanoparticle rods and the magnetic gradient. In certain embodiments, the circulating or oscillatory motion of the magnetic nanoparticles increases exposure of a targeted structure (e.g. a fluid obstruction) within a blood vessel of the vasculature to a therapeutic agent (e.g. a thrombolytic drug) present in the blood vessel and accelerates action of the therapeutic agent (e.g. the thrombolytic drug on the fluid obstruction).

In some embodiments, the systems comprise various features that are present as single features (as opposed to multiple features). For example, the systems may consist of a single permanent magnet as opposed to multiple magnets. In some embodiments, the systems comprise one or more of the following: means for controlling rotation of a magnet, means for delivering magnetic particles (e.g., intravenous or intra-arterial infusion assembly or intravascular catheter), etc.”

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. The headings used herein are merely provided to enhance readability and are not intended to limit the scope of the embodiments disclosed in a particular section to the features or elements disclosed in that section.

While the embodiments disclosed herein are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “advancing a catheter” include “instructing advancing a catheter.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 1000 nm” includes “1000 nm.” Terms or phrases preceded by a term such as “substantially” or “generally” include the recited term or phrase. For example, “substantially continuously” includes “continuously.”

What is claimed is:

1. A method of facilitating treatment of a clot within a blood vessel of a subject, the method comprising:
advancing a distal end of a microcatheter to a location proximal to, distal to or within the clot;
delivering magnetic particles through the microcatheter at the location of the clot;
delivering a thrombolytic agent through the microcatheter at the location of the clot;
deploying a thrombectomy device from the distal end of the microcatheter so as to capture the clot with the thrombectomy device;
applying a rotating magnetic field so as to cause the magnetic particles to agglomerate into stir bars and to travel in a rotating motion;
withdrawing the thrombectomy device from the blood vessel of the subject,
wherein the stir bars and the thrombolytic agent are adapted and positioned to travel with, and facilitate lysis of, any clot fragments that break off from the clot during the deploying and withdrawing of the thrombectomy device.

2. The method of claim 1, wherein delivering the thrombolytic agent through the microcatheter at the location of the clot comprises delivering the thrombolytic agent at locations proximal to, within, and/or distal of the clot.

3. The method of claim 1, wherein applying the rotating magnetic field comprises rotating a permanent magnet positioned external to the subject.

4. The method of claim 3, further comprising adjusting a position and/or orientation of the permanent magnet so as to cause the stir bars to travel to a desired location.

5. The method of claim 3, wherein rotating the permanent magnet comprises rotating the permanent magnet at a frequency of between 1 Hz and 10 Hz, and wherein a magnitude of the magnetic field is between 0.01 and 1 Tesla.

6. The method of claim 5, wherein the frequency is between 3 Hz and 6 Hz.

7. The method of any of claims 1-6, further comprising delivering a diagnostic agent through the microcatheter at the location of the clot.

8. The method of claim 7, wherein the diagnostic agent comprises a contrast agent.

9. The method of claim 7, wherein the diagnostic agent is attached to one or more of the magnetic particles.

10. The method of any of claims 1-6, further comprising imaging the magnetic particles utilizing an imaging modality.

11. The method of claim 10, wherein the imaging modality is a magnetic resonance imaging modality.

12. The method of claim 10, wherein the imaging modality is an ultrasound-based imaging modality.

13. The method of any of claims 1-6, further comprising imaging the stir bars using fluoroscopy.

14. The method of any of claims 1-6, wherein advancing the distal end of the microcatheter to the location of the clot comprises positioning a distal terminus of the microcatheter at a location distal of the clot.

15. The method of any of claims 1-6, wherein advancing the distal end of the microcatheter to the location of the clot comprises advancing the microcatheter over a guidewire.

16. The method of any one of claims 1-6, wherein the blood vessel is a cerebral artery.

17. The method of any of claims 1-6, wherein the blood vessel is a peripheral artery.

18. The method of any of claims 1-6, wherein the blood vessel is within a leg or an arm.

19. The method of claim 18, wherein the blood vessel is a vein.

20. The method of claim 18, wherein the blood vessel is an artery.

21. The method of any of claims 1-6, wherein the magnetic particles comprise magnetite nanoparticles.

22. A method of facilitating treatment of an obstruction within a lumen of a subject, the method comprising:
advancing a distal end of a microcatheter to a location proximal to, distal to or at a location of the obstruction;
delivering magnetic particles through the microcatheter at the location of the obstruction;
delivering a therapeutic agent through the microcatheter at the location of the obstruction;
deploying a retrieval device from the distal end of the microcatheter so as to remove the obstruction with the retrieval device;
applying a rotating magnetic field so as to cause the magnetic particles to agglomerate into stir bars and to travel in a rotating motion;
withdrawing the retrieval device from the lumen of the subject,
wherein the stir bars and the therapeutic agent are adapted and positioned to travel with, and facilitate lysis of, any fragments that break off from the obstruction during the deploying and withdrawing of the retrieval device.

23. The method of claim 22, wherein delivering the therapeutic agent through the microcatheter at the location of the obstruction comprises delivering the therapeutic agent at locations proximal to, within, and/or distal of the obstruction.

24. The method of claim 22, wherein applying the rotating magnetic field comprises rotating a permanent magnet positioned external to the subject.

25. The method of claim 24, further comprising adjusting a position and/or orientation of the permanent magnet so as to cause the stir bars to travel to a desired location.

26. The method of claim 24, wherein rotating the permanent magnet comprises rotating the permanent magnet at a frequency of between 1 Hz and 10 Hz, and wherein a magnitude of the magnetic field is between 0.01 and 1 Tesla.

27. The method of claim 26, wherein the frequency is between 3 Hz and 6 Hz.

28. The method of any of claims 22-27, further comprising delivering a diagnostic agent through the microcatheter at the location of the obstruction.

29. The method of claim 28, wherein the diagnostic agent comprises a contrast agent.

30. The method of claim 28, wherein the diagnostic agent is attached to one or more of the magnetic particles.

31. The method of any of claims 22-27, further comprising imaging the magnetic particles utilizing an imaging modality.

32. The method of claim 31, wherein the imaging modality is a magnetic resonance imaging modality.

33. The method of claim 31, wherein the imaging modality is an ultrasound-based imaging modality.

34. The method of any of claims 22-27, further comprising imaging the stir bars using fluoroscopy.

35. The method of any of claims 22-27, wherein advancing the distal end of the microcatheter to the location of the obstruction comprises positioning a distal terminus of the microcatheter at a location distal of the obstruction.

36. The method of any of claims 22-27, wherein advancing the distal end of the microcatheter to the location of the obstruction comprises advancing the microcatheter over a guidewire.

37. The method of any of claims 22-27, wherein the lumen is a cerebral artery.

38. The method of any of claims 22-27, wherein the lumen is a peripheral artery.

39. The method of any of claims 22-27, wherein the lumen is a blood vessel within a leg or an arm of the subject.

40. The method of claim 39, wherein the blood vessel is a vein.

41. The method of claim 39, wherein the blood vessel is an artery.

42. The method of any of claims 22-27, wherein the obstruction is a tumor.

43. The method of any of claims 22-27, wherein the obstruction is plaque.

44. The method of any of claims 22-27, wherein the magnetic particles comprise magnetite nanoparticles.

45. A system adapted to facilitate thrombectomy procedures with enhanced efficacy and safety, the system comprising:
a multi-lumen microcatheter comprising a primary lumen and a secondary lumen, the outer diameter of the microcatheter being sized to fit within a cerebral blood vessel of a subject;
a thrombectomy device adapted to be delivered through the primary lumen of the multi-lumen microcatheter, the thrombectomy device comprises an expandable member adapted to capture and retrieve a clot;
a permanent magnet adapted to be positioned external to the subject;
a magnetic controller adapted to rotate the permanent magnet so as to cause magnetic particles introduced within the secondary lumen of the microcatheter to agglomerate into stir bars and to travel in a rotating end-over-end motion,
wherein the rotating end-over-end motion of the stir bars causes one or more therapeutic agents delivered through the catheter to remain at the location of the clot.

46. A method of facilitating treatment and/or diagnosis of a therapeutic and/or diagnostic target within a body of a subject, the method comprising:
advancing a distal end of a catheter to a location of the target within the body of the subject;
introducing a plurality of magnetic particles within a lumen of the catheter;
applying a rotating magnetic field using a permanent magnet positioned external to the subject so as to cause the magnetic particles to agglomerate into stir bars within the lumen of the catheter;
imaging the stir bars within the lumen of the catheter;
determining a desired direction of travel of the stir bars based on the imaging;
adjusting at least one of a position and an orientation of the permanent magnet so as to cause the stir bars to travel in the desired direction of travel;
delivering the stir bars and one or more therapeutic, diagnostic, and/or theranostic agents out of the catheter toward the location of the target; and
continuously applying the rotating magnetic field for a duration of time such that the stir bars convey the one or more therapeutic, diagnostic, and/or theranostic agents to the target.

47. The method of claim 46, wherein imaging the stir bars comprises use of at least one of fluoroscopy, ultrasound-based imaging, magnetic resonance imaging and tomography-based imaging.

48. The method of claim 46 or 47, wherein the catheter comprises a microcatheter.

49. The method of claim 46 or 47, wherein the magnetic particles comprise magnetite nanoparticles.

50. The method of claim 46 or 47, wherein the magnetic particles comprise superparamagnetic particles.

51. The method of claim 46 or 47, wherein the magnetic particles comprise ferrimagnetic particles.

52. The method of any of claims 46-51 wherein the target is a tumor and wherein the one or more therapeutic, diagnostic, and/or theranostic agents comprise a drug adapted to treat the tumor.

53. The method of any of claims 46-51, wherein the target is an obstruction within a Fallopian tube.

54. The method of claim 46 or 47, wherein the target is a vascular occlusion in a vessel of a leg or arm of the subject.

55. The method of claim 46, wherein the one or more therapeutic, diagnostic, and/or theranostic agents comprise a neuroprotectant.

56. The method of claim 46, wherein the one or more therapeutic, diagnostic, and/or theranostic agents comprise a contrast agent.

57. A method of facilitating treatment of a therapeutic target within a body of a subject, the method comprising:
advancing a distal end of a catheter to a location of the therapeutic target within the body of the subject;
introducing a plurality of magnetic particles within a lumen of the catheter;
applying a rotating magnetic field so as to cause the magnetic particles to agglomerate into stir bars within the lumen of the catheter;
delivering the stir bars and one or more therapeutic agents out of the catheter toward the location of the therapeutic target;
continuously applying the rotating magnetic field for a duration of time such that the stir bars convey the one or more therapeutic agents to the therapeutic target, thereby facilitating treatment of the therapeutic target.

58. The method of claim 57, wherein the magnetic particles comprise magnetite nanoparticles.

59. The method of claim 57, wherein applying the rotating magnetic field and continuously applying the rotating magnetic field comprise causing a permanent magnet positioned external to the subject to be rotated at a frequency of between 1 Hz and 10 Hz, and wherein a magnitude of the magnetic field is between 0.01 and 1 Tesla.

60. The method of claim 59, wherein the frequency is between 3 Hz and 6 Hz.

61. The method of any of claims 57 to 60, wherein the therapeutic target is a clot within a blood vessel.

62. The method of any of claims 57 to 60, wherein the therapeutic target is a vascular occlusion within a vessel of the subject.

63. The method of claim 62, wherein the vessel is within an extremity of the subject.

64. The method of claim 63, wherein the extremity is a leg or an arm.

65. The method of claim 62, wherein the vessel is a peripheral artery.

66. The method of any of claims 63 to 65, wherein the vessel is a vein.

67. The method of claim 61 or 62, wherein the one or more therapeutic agents comprise one or more thrombolytic drugs.

68. The method of claim 61, wherein the one or more therapeutic agents comprise a neuroprotectant.

69. The method of any of claims 57 to 60, wherein the therapeutic target is a tumor.

70. The method of any of claims 57 to 60, further comprising delivering one or more diagnostic agents through the catheter.

71. The method of claim 70, wherein the one or more diagnostic agents comprise a contrast agent.

72. The method of any of claims 57 to 60, wherein the one or more therapeutic agents comprise one or more theranostic agents.

73. A method of facilitating treatment and/or diagnosis of a therapeutic and/or diagnostic target within a body of a subject, the method comprising:
delivering magnetic particles to a location near the target within the body of the subject;
applying a rotating magnetic field so as to cause the magnetic particles to agglomerate into stir bars and to travel in a rotating motion toward the therapeutic target;
delivering one or more theranostic agents to the location near the target;
wherein the travel of the stir bars in a rotating motion causes the one or more theranostic agents to reach the target and to remain at the target.

74. The method of claim 73, wherein:
the magnetic particles comprise magnetite nanoparticles, applying the rotating magnetic field comprises rotating a permanent magnet positioned external to the body of the subject at a frequency of between 3 Hz and 6 Hz,
a magnitude of the magnetic field is between 0.01 and 1 Tesla, and
delivering the magnetic particles and delivering the one or more theranostic agents comprises local delivery through a catheter to the location near the target.

75. The method of claim 73, wherein applying the rotating magnetic field comprises rotating a permanent magnet positioned external to the body of the subject at a frequency of between 0.1 Hz and 100 Hz, and wherein a magnitude of the magnetic field is between 0.01 and 1 Tesla.

76. The method of claim 75, wherein the frequency is between 1 Hz and 10 Hz.

77. The method of claim 73, wherein delivering the magnetic particles and delivering the one or more theranostic agents comprises local delivery through a catheter to the location near the target.

78. The method of any of claims 73-77, wherein the target is a tumor and wherein the one or more theranostic agents comprise a drug adapted to treat the tumor.

79. The method of any of claims 73-77, wherein the target is an obstruction within a Fallopian tube.

80. The method of any of claims 73-77, wherein the target is at an intraosseous location.

81. The method of any of claims 73-77, wherein the target is a vascular occlusion in a vessel of a leg or arm of the subject.

82. The method of any of claims 73-77, wherein the target is an obstruction within an in-dwelling catheter positioned within the body of the subject.

83. The method of any of claims 73-77, wherein the one or more theranostic agents is attached to one or more of the magnetic particles.

84. The method of any of claims 73-83, wherein delivering the magnetic particles and delivering the one or more theranostic agents comprises systemic intravenous delivery.

85. The method of claim 84, wherein the magnetic particles and the one or more theranostic agents are co-administered together at the same time.

86. The method of claim 84, wherein the magnetic particles and the one or more theranostic agents are delivered separately.

87. The method of claim 73, wherein applying the rotating magnetic field comprises controlling currents within an electromagnet.

88. The method of claim 73, wherein applying the rotating magnetic field comprises rotating multiple permanent magnets positioned external to the body of the subject.

89. A method of facilitating clearance of an obstruction within an in-dwelling catheter without removing the catheter from a subject, the method comprising:
introducing a plurality of magnetic particles within a lumen of the catheter;
applying a rotating magnetic field using a permanent magnet positioned external to the subject so as to cause the magnetic particles to agglomerate into stir bars and to travel in a rotating end-over-end motion toward the obstruction; and
delivering one or more therapeutic agents adapted to clear the obstruction into the lumen of the catheter,
wherein the rotating end-over-end motion of the stir bars causes the one or more therapeutic agents to be conveyed to the obstruction such that the one or more the therapeutic agents can effectively clear the obstruction.

90. The method of claim 89, further comprising imaging the stir bars or the motion of the stir bars so as to identify a location of the obstruction.

91. A method of facilitating clearance of an obstruction within an in-dwelling catheter without removing the catheter from a subject, the method comprising:
introducing a plurality of magnetic particles within a lumen of the catheter;
applying a rotating magnetic field using a permanent magnet positioned external to the subject so as to cause the magnetic nanoparticles to agglomerate into stir bars and to travel in a rotating end-over-end motion toward the obstruction; and
imaging the stir bars or the motion of the stir bars so as to identify a location of the obstruction.

92. The method of claim 91, further comprising clearing the obstruction based on identified location.
